(19) United States
(12) Patent Application Publication
(10) Pub. No.: US 2026/0266849 A1
(43) Pub. Date: Sep. 10, 2026

(54) METHODS AND SYSTEMS FOR SERIAL AMPLIFICATION OF TAU ASSEMBLIES

(71) Applicant: University of Denver, Denver, CO (US)

(72) Inventors: Martin Margittai, Englewood, CO (US); Jasdeep Singh, Denver, CO (US); Zachariah Yasir Gabani, Littleton, CO (US)

(21) Appl. No.: 19/535,901

(22) Filed: Feb. 10, 2026

Related U.S. Application Data
(60) Provisional application No. 63/757,281, filed on Feb. 11, 2025.

(51) Int. Cl. G01N 33/68

(52) U.S. Cl. CPC G01N 33/6896; G01N 2800/2821

(57) ABSTRACT

Methods for serial amplification of Tau filaments and Tau assemblies include contacting monomeric Tau protein with Tau seed material under conditions permissive for seed-dependent fibril or assembly growth to produce amplified Tau filaments or assemblies, processing at least a portion of the amplified material to generate Tau seed material for further amplification, and repeating the contacting and processing steps to produce serially amplified Tau filaments or assemblies, where the monomeric Tau includes one or more Tau isoforms, variants, fragments, or modified forms. In some embodiments, Tau seed material is obtained from a biological sample associated with a Tauopathy. The methods further include characterizing amplified Tau filaments or assemblies or using the serial amplification process to identify compounds that modulate Tau assembly propagation by comparing propagation characteristics in the presence and absence of a test compound.

### REFERENCE TO RELATED APPLICATIONS

The present invention claims the benefit of U.S. Provisional Patent Application No. 63/757,281, filed on Feb. 11, 2025, and titled “Methods and associated systems for generating pathological Tau filaments in absence of co-factors,” which is incorporated hereby in its entirety by reference.

### FIELD OF THE DISCLOSURE

The present disclosure relates generally to protein aggregation and propagation technologies. In particular, but not by way of limitation, the present disclosure relates to methods and systems for generating, amplifying, and characterizing aggregated protein assemblies associated with neurodegenerative diseases.

### DESCRIPTION OF RELATED ART

Tau filaments (also referred to as fibrils) are the pathological hallmark of Alzheimer's disease (AD) and more than twenty other fatal neurodegenerative disorders. In particular, AD is known to be characterized by the presence of amyloid-beta (Aβ) plaques and Tau neurofibrillary tangles (NFTs).

### BRIEF DESCRIPTION OF THE DRAWINGS

FIG. 1 graphically shows illustrative steps involved in a multistep Tau amplification assay from a tissue extract, in accordance with various embodiments disclosed herein.

FIG. 2 is a process flow chart illustrating a multistep Tau amplification assay from a tissue extract, in accordance with various embodiments disclosed herein.

FIG. 3A shows experimental results from Step 1 of the multistep Tau amplification assay of FIGS. 1 and 2, shown here to illustrate the formation of Tau fibrils from brain extract of a first Alzheimer's Disease patient (AD14), in accordance with various embodiments disclosed herein.

FIG. 3B shows experimental results from Step 1 of the multistep Tau amplification assay of FIGS. 1 and 2, shown here to illustrate the formation of Tau fibrils from brain extract of a second Alzheimer's Disease patient (AD53), in accordance with various embodiments disclosed herein.

FIG. 3C shows experimental results from Step 1 of the multistep Tau amplification assay of FIGS. 1 and 2, shown here to illustrate the formation of Tau fibrils from brain extract of a third Alzheimer's Disease patient (AD71), in accordance with various embodiments disclosed herein.

FIG. 4 shows a bar graph illustrating the quantified SDS-PAGE and Coomassie staining results of FIGS. 3A-3C, in accordance with various embodiments disclosed herein.

FIG. 5 shows transmission electron microscopy (TEM) images illustrating the formation of disease fibrils from brain tissue extract as corresponding to the results shown in FIGS. 3A-3C, in accordance with various embodiments disclosed herein.

FIG. 6A shows experimental results from Steps 2 and 3 of the multistep Tau amplification assay of FIGS. 1 and 2, shown here to further illustrate the formation of Tau fibrils from diseased brain extract, in accordance with various embodiments disclosed herein. FIG. 6B shows experimental results from Steps 2 and 3 of the multistep Tau amplification assay of FIGS. 1 and 2, shown here to further illustrate the formation of Tau fibrils from diseased brain extract, in accordance with various embodiments disclosed herein.

FIG. 7A shows experimental results from Steps 4 and 5 of the multistep Tau amplification assay of FIGS. 1 and 2, shown here to further illustrate the formation of Tau fibrils from diseased brain extract, in accordance with various embodiments disclosed herein. FIG. 7B shows experimental results from Steps 4 and 5 of the multistep Tau amplification assay of FIGS. 1 and 2, shown here to further illustrate the formation of Tau fibrils from diseased brain extract, in accordance with various embodiments disclosed herein.

FIG. 8 shows experimental results from Step 6 of the multistep Tau amplification assay of FIGS. 1 and 2, shown here to illustrate the formation of Tau fibrils from diseased brain extract, in accordance with various embodiments disclosed herein.

FIG. 9 shows TEM images illustrating the formation of disease fibrils from brain tissue extract as corresponding to the results shown in FIG. 8 in accordance with various embodiments disclosed herein.

FIG. 10 shows bar graphs illustrating the quantified SDS-PAGE and Coomassie staining results of FIGS. 6-8 in accordance with various embodiments disclosed herein.

FIG. 11 shows experimental results from Step 1 of the multistep Tau amplification assay of FIGS. 1 and 2, shown here to illustrate the formation of Tau fibrils from brain extract of three Pick's Disease patients, in accordance with various embodiments disclosed herein.

FIG. 12 shows a bar graph illustrating the quantified SDS-PAGE and Coomassie staining results of FIG. 11, in accordance with various embodiments disclosed herein.

FIG. 13 shows TEM images illustrating the formation of disease fibrils from brain tissue extract as corresponding to the results shown in FIG. 11, in accordance with various embodiments disclosed herein.

FIG. 14 shows experimental results from Step 1 of the multistep Tau amplification assay of FIGS. 1 and 2, shown here to illustrate the formation of Tau fibrils from brain extract of three Progressive Supranuclear Palsy patients, in accordance with various embodiments disclosed herein.

FIG. 15 shows a bar graph illustrating the quantified SDS-PAGE and Coomassie staining results of FIG. 14, in accordance with various embodiments disclosed herein.

FIG. 16 shows TEM images illustrating the formation of disease fibrils from brain tissue extract as corresponding to the results shown in FIG. 14, in accordance with various embodiments disclosed herein.

FIG. 17 shows a system suitable for performing the multistep Tau amplification assay, in accordance with various embodiments disclosed herein.

FIG. 18A shows experimental results demonstrating serial amplification of Alzheimer's disease (AD)-derived Tau assemblies using recombinant 3R Tau monomeric substrate across multiple amplification generations, in accordance with various embodiments disclosed herein. FIG. 18B shows experimental results demonstrating serial amplification of Alzheimer's disease (AD)-derived Tau assemblies using recombinant 3R Tau monomeric substrate across multiple amplification generations, in accordance with various embodiments disclosed herein. FIG. 18C shows experimental results demonstrating serial amplification of Alzheimer's disease (AD)-derived Tau assemblies using recombinant 3R Tau monomeric substrate across multiple amplification generations, in accordance with various embodiments disclosed herein. FIG. 18D shows experimental results demonstrating serial amplification of Alzheimer's disease (AD)-derived Tau assemblies using recombinant 3R Tau monomeric substrate across multiple amplification generations, in accordance with various embodiments disclosed herein.

FIGS. 19A-1-19C show experimental results demonstrating serial amplification of Alzheimer's disease (AD)-derived Tau assemblies using recombinant 3R Tau monomeric substrates, 4R Tau monomeric substrates, and combinations of 3R and 4R Tau monomeric substrates across multiple amplification generations, in accordance with various embodiments disclosed herein.

FIGS. 20A-20B show experimental results demonstrating cross-seeding behavior of serially amplified Tau assemblies, including recruitment of 3R Tau onto 4R Tau assemblies and recruitment of 4R Tau onto 3R Tau assemblies, in accordance with various embodiments disclosed herein.

FIGS. 21A-21D show experimental results demonstrating that Tau assemblies serially amplified from Alzheimer's disease (AD)-derived Tau seed material induce intracellular Tau aggregation in a cell-based assay, in accordance with various embodiments disclosed herein.

FIGS. 22A-1-22C show experimental results demonstrating sensitivity of co-factor-free, seed-dependent Tau assembly growth to reaction conditions, including ionic strength dependence, oxidation-state dependence, and seed dependence, in accordance with various embodiments disclosed herein.

FIG. 23A-23D shows experimental results characterizing Tau seed preparation for serial amplification, including fibril fragmentation and seed length distribution, in accordance with various embodiments disclosed herein.

FIGS. 24A-24C-2 show experimental results demonstrating amplification behavior of Tau assemblies derived from non-AD tauopathies, including Pick's disease (PiD) and progressive supranuclear palsy (PSP), and associated isoform-specific recruitment characteristics, in accordance with various embodiments disclosed herein.

FIG. 25A-25H shows experimental results demonstrating seeded amplification of Tau assemblies using Alzheimer's disease (AD)-derived Tau seed material and recombinant Tau monomeric substrates under co-factor-free conditions, in accordance with various embodiments disclosed herein.

FIGS. 26A-1-26D-5 shows experimental results demonstrating serial propagation of Tau assemblies over multiple amplification generations and associated conversion metrics, in accordance with various embodiments disclosed herein.

FIG. 27 shows experimental results demonstrating isoform-specific recruitment behavior of Tau assemblies derived from AD, in accordance with various embodiments disclosed herein.

FIG. 28 shows experimental results demonstrating that Tau fibrils amplified from AD brain homogenates induce intracellular Tau aggregation in a cell-based assay, in accordance with various embodiments disclosed herein.

FIGS. 29A-29F show experimental results demonstrating structural or ultrastructural characterization of serially amplified Tau assemblies, in accordance with various embodiments disclosed herein.

FIGS. 30A-30H show experimental results demonstrating functional activity of serially amplified Tau assemblies in a biological assay system, in accordance with various embodiments disclosed herein.

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents.

### DETAILED DESCRIPTION

Understanding of the mechanisms behind pathological aggregation of the protein Tau may be important in diagnosing and treating tauopathy-based neurodegenerative disorders. Efforts to characterize Tau filament formation and aggregation characteristics have been hampered by the fact that generation and propagation of Tau filaments and their aggregates are exceedingly difficult in a laboratory setting.

In some aspects, prior techniques have relied on co-factors, such as heparin or dextran sulfate, as an inducer. However, the use of co-factors is known to modify the resulting Tau filament structure from those found in patients with tauopathies, thus limiting the usefulness of the Tau filaments so generated in studies.

Filaments composed of the microtubule-associated protein Tau are a pathological hallmark of Alzheimer's disease (AD) and over twenty other neurodegenerative disorders, collectively known as tauopathies. In the adult human brain, six Tau isoforms are expressed due to alternative mRNA splicing from a single gene (MAPT) on chromosome 17. Tau isoforms can range from about 352 to about 441 amino acids in length and can differ with respect to (i) the number of N-terminal inserts and (ii) the complement of C-terminal microtubule-binding repeat domains. For example, one microtubule-binding repeat (commonly referred to as repeat 2) may be absent or present, thereby yielding three-repeat (3R) Tau isoforms or four-repeat (4R) Tau isoforms, respectively.

Individual repeats are 31-32 residues in length and span residues 244 to 368 within the protein. Alzheimer's disease is a mixed tauopathy, in which all Tau isoforms are found in the filaments. In progressive supranuclear palsy (PSP) there is preferential deposition of 4R Tau, making it a four-repeat tauopathy. In Pick's disease (PiD), there is preferential deposition of 3R Tau, making it a three-repeat tauopathy.

Tau monomers are intrinsically disordered, but when assembled into fibrils, the repeat regions form a parallel and in-register β-sheet structure, in which identical residues from neighboring proteins are perfectly stacked on top of each other. Although these early insights were gained from fibrils that were formed in vitro, pathological Tau filaments proved to share the in-register arrangement of β-strands. The cryo-electron microscopy analysis of Tau filaments isolated from AD brain provided the first high resolution structure. The filaments, which are predominantly of the paired helical filament type, were observed to have a cross-β/β-helical fold that encompasses residues 304/306-378/380 in repeats 3 and 4 and the immediately adjacent C-terminal region. These residues are common to all Tau isoforms and provide an explanation for their joint incorporation into the filament. Residues outside the structured region form a fuzzy, disordered coat that surrounds the central core. Filaments in PiD and PSP were found to have β-sheet folds that differ from the one in AD involving residues 254-378 and 272-381, respectively. Structural evidence from other diseases corroborated that each tauopathy is characterized by a specific fold.

The pathology of Tau spreads transsynaptically from one neuron to another, as well as from neurons to other cell types such as oligodendrocytes and spatiotemporally via defined pathways throughout the brain, suggesting that small Tau aggregates serve as seeds that recruit naïve Tau monomers onto their ends. This structural conversion may be observed in multiple in vivo model systems and together with the observation of specific Tau folds indicates that the protein shares key properties with prions. However, problematically, the molecular mechanisms of seeded (or templated) Tau aggregation remain poorly resolved. It has been unknown how different environmental conditions modulate Tau recruitment onto the fibril end.

Negatively charged co-factors may greatly enhance the spontaneous aggregation of Tau protein in vitro. The use of the co-factor heparin also facilitated the amplification of minute quantities of pathological fibrils from AD and other tauopathies. These assays mechanically fracture Tau fibrils into smaller seeds to then recruit either full-length or truncated monomers into the fibrils. More recently, sarkosyl-insoluble material from AD brain was successfully used to amplify full-length Tau in the absence of co-factors. These Generation 1 fibrils possessed the paired helical filament structure of the original fibrils, but surprisingly they lacked the ability to initiate seed amplification of another generation. In various aspects, this suggested that co-factors or other components may be needed to sustain pathological fibril growth. In support of this, RNA and heparan sulfate are found in AD-filament containing lesions in AD. Furthermore, co-factors were necessary for templated aggregation of synthetic Tau fibrils. Additionally, fibrils that were generated on a platform in which heparin was immobilized to the surface, required co-factor for further seeding, although the fibrils had no co-factor incorporated.

Contrasting these findings, a prior technique demonstrated that the core region of Tau is sufficient to form paired helical filaments without the addition of co-factors. Even more, it was shown that filaments composed of the core region were able to recruit full-length Tau. These seemingly contradictory findings raise the question as to what determines whether full-length Tau monomers can or cannot be recruited into the fibril. Various embodiments of the present disclosure advantageously address this question. In various aspects, some embodiments disclosed herein show that filaments from AD, PSP, and PiD can be serially amplified in the absence of co-factors and in low salt buffer, but that physiological salt concentrations are inhibitory. These illustrative findings demonstrate that Tau protein alone encodes all the information for fibril propagation and imply that the conformational status of the Tau monomer ensemble dictates whether templated growth proceeds.

Thus, there is a need for improved methods and associated systems for generation and amplification of full-length Tau filaments.

In various embodiments, methods for generating and amplifying Tau filaments without co-factors are disclosed. In embodiments, the methods include serial (also referred to as multistep) amplification of pathological filaments. As used herein, the terms “Tau assemblies,” “assemblies,” “Tau assembly,” and “assembly,” refer to aggregated Tau species such as fibrils and filaments capable of templated propagation. As used herein, “Tau assemblies” include Tau filaments, Tau fibrils, and other aggregated Tau species capable of templated propagation, unless context indicates otherwise.

In some embodiments, the methods include using pathological Tau filaments as seeds for the generation of full-length Tau filaments therefrom.

In some embodiments, the methods include using recombinant Tau protein for fibril elongation.

In some embodiments, the methods include facilitating serial amplification of the Tau filaments in a template-assisted manner, in the absence of co-factors.

In some embodiments, a fibril structure of the pathological Tau filaments is preserved during the amplification.

In various embodiments, associated systems for generating and amplifying Tau filaments without co-factors are disclosed.

In various embodiments, methods for serial amplification of Tau filaments are disclosed. The methods include incubating monomeric Tau protein with Tau fibril seeds under conditions that support fibril elongation and their subsequent amplification. The Tau fibril seeds may be pathological or other Tau fibril seeds. The method further includes generating amplified Tau fibrils from the monomer-seed mixture, and isolating, diluting, or transferring the amplified Tau fibrils to initiate at least one subsequent amplification round. The methods include repeating the incubating, generating, and isolating steps for one or more amplification cycles. In embodiments, the monomeric Tau includes one or more isoforms selected from 3R Tau, 4R Tau, 0N/1N/2N variants, truncated Tau fragments, recombinant constructs, post-translationally modified Tau, or disease-associated splice variants. The method yields serially amplified Tau fibrils suitable for biochemical, structural, or functional characterization.

In some embodiments, the Tau fibril seeds may be selected from Alzheimer's disease (AD) Tau, Pick's disease (PiD) Tau, progressive supranuclear palsy (PSP) Tau, corticobasal degeneration (CBD) Tau, chronic traumatic encephalopathy (CTE) Tau, argyrophilic grain disease (AGD) Tau, globular glial tauopathy (GGT) Tau, or Tau polymorphs from any other disease (e.g., any tauopathy).

In some embodiments, the monomeric Tau includes 3R-only isoforms, 4R-only isoforms, combinations thereof, or recombinant constructs expressing disease-relevant repeat domains.

In some embodiments, the amplification is performed for 2-50 rounds.

In some embodiments, successive amplification cycles include dilution of Tau seed material such that non-Tau components originating from a biological source of the seed material are progressively reduced. In such embodiments, after multiple amplification cycles, the serially amplified Tau filaments are substantially free of non-Tau proteins, nucleic acids, lipids, or other biological components present in the original biological sample, while retaining seed-dependent propagation activity.

In some embodiments, the amplification occurs under shaking, agitation, quiescent incubation, sonication, mechanical cycling, temperature cycling, or RT-QuIC-like conditions.

As used herein, the terms “seed material,” “Tau seed material,” and “seed” refer to any material comprising Tau species (e.g., including but not limited to Tau monomers and aggregated Tau species) capable of inducing or templating conversion of monomeric Tau into aggregated Tau assemblies under conditions permissive for seed-dependent Tau assembly growth. In some embodiments, seed material comprises one or more Tau assemblies, including but not limited to Tau filaments, Tau fibrils, fibril fragments, protofibrils, oligomers, or other aggregated Tau conformers that retain seeding competence. Seed material may be obtained from a biological sample (e.g., tissue, homogenate, cerebrospinal fluid, or blood-derived extracellular vesicles) and/or may be generated in vitro, including as products of one or more amplification reactions described herein.

In some embodiments, seed material is subjected to processing to increase seeding efficiency, for example by agitation, shear, quaking, or sonication to generate a population of seed-competent fragments. In some embodiments, seed material is prepared, stored, and/or distributed as one or more aliquots, including diluted aliquots (including serial dilutions), optionally stored in frozen, refrigerated, lyophilized, or otherwise stabilized form, and optionally prepared (e.g., thawed or reconstituted) prior to use.

As used herein, “Tau fibril seed material” refers to seed material comprising Tau fibrils and/or Tau filaments, including fragments thereof, that provide growth-competent ends capable of recruiting monomeric Tau into fibrillar or filamentous Tau assemblies. In some embodiments, Tau fibril seed material comprises pathological Tau fibrils derived from tauopathy-associated biological material and/or Tau fibrils generated in vitro by seed-dependent amplification using recombinant Tau monomeric substrate, including serially amplified Tau fibrils produced according to embodiments described herein.

In some embodiments, Tau fibril seed material comprises an aliquot obtained from an amplification generation (e.g., Generation 1, Generation 2, Generation 3, Generation 4, Generation 5, Generation 6, or later) and used to seed a subsequent amplification reaction, including use as a diluted aliquot prepared from a prior generation product.

In some embodiments, “conditions permissive for seed-dependent Tau assembly growth” or “conditions permissive for seed-dependent fibril elongation” refer to reaction conditions that, in the presence of Tau seed material, result in detectable conversion of monomeric Tau into filamentous or fibrillar Tau assemblies. Such conditions may be identified by one or more readouts including but not limited to increased Thioflavin T (ThT) signal, increased sedimentable Tau in a pellet fraction following centrifugation, visualization of filamentous structures by electron microscopy, or seeding activity in a cell-based assay.

In some embodiments, conditions permissive for seed-dependent Tau assembly growth include one or more of: a buffered aqueous solution (for example, a Tris-, phosphate-, HEPES-, or similar buffer) having a pH in a physiological or near-physiological range; a defined ionic strength selected to support monomer solubility and seed-templated recruitment; a selected monomeric Tau concentration sufficient to permit elongation on the provided seeds; and incubation at a temperature selected to promote elongation (for example, room temperature to about 45 degrees Celsius (° C.), including about 37° C.). In some embodiments, permissive conditions include addition of a reporter dye such as ThT for monitoring kinetics. In some embodiments, permissive conditions exclude addition of negatively charged aggregation-promoting co-factors such as heparin, dextran sulfate, RNA, or other polyanionic inducers.

In some embodiments, the serial amplification methods disclosed herein are performed in the absence of added negatively charged aggregation-promoting co-factors, including heparin, dextran sulfate, RNA, or synthetic polyanions, such that Tau assembly growth is driven by seed-dependent templating rather than co-factor-induced aggregation.

In some embodiments, permissive conditions include application of intermittent or periodic agitation to facilitate mixing and/or controlled fragmentation of assemblies, including shaking, quaking, orbital agitation, tapping, or other mechanical cycling, optionally interleaved with quiescent incubation periods. In some embodiments, permissive conditions additionally or alternatively include controlled fragmentation by sonication (including bath or probe sonication) or other mechanical disruption to generate daughter seeds for subsequent amplification cycles. The particular selection of buffer composition, ionic strength, monomer concentration, temperature, agitation regime, and cycle duration may be optimized depending on the tauopathy source of the seed material, the monomeric Tau isoform or construct used as substrate, and the desired propagation outcome.

In some embodiments, the amplified fibrils preserve at least one structural or functional feature of the parental or original seed, including width, periodicity, twist morphology, core domain architecture, or absence or presence of seeding barriers.

In some embodiments, the amplified fibrils exhibit structural changes relative to the parental seed.

In some embodiments, characterization of the amplified Tau fibrils includes one or more of Thioflavin-T or Thioflavin-S fluorescence kinetics, luminescent-conjugated oligothiophene binding, biochemical sedimentation or ultracentrifugation, protease digestion profiling, mass spectrometry, electron microscopy, atomic force microscopy, or cell-based seeding or biosensor assays.

In some embodiments, the amplification is conducted in a microplate, tube, perfusion chamber, continuous-flow system, microfluidic device, or automated amplification device.

In some embodiments, the monomeric Tau is phosphorylated, acetylated, ubiquitinated, truncated, recombinant, or chemically modified.

In various embodiments, multimodal analytical platforms for analyzing Tau fibril propagation during serial amplification are disclosed. The platform includes a fluorescence-based kinetic module for detecting Tau fibril elongation, a biochemical sedimentation or fractionation module for quantifying monomer-to-fibril conversion, an electron microscopy imaging module for evaluating fibril morphology or ultrastructure, a cellular assay module for measuring Tau internalization, seeding, or propagation, and a computational module configured to analyze propagation characteristics across amplification rounds.

In some embodiments, the kinetic module of the platform uses Thioflavin-T fluorescence.

In some embodiments, the electron microscopy imaging module includes negative-stain EM, tomography, or cryo-electron microscopy.

In some embodiments, the computational module generates a disease-specific propagation fingerprint or Tau strain classification.

In some embodiments, the platform further includes a machine-learning module configured to distinguish Tau strains based on multimodal amplification data.

In various embodiments, methods for characterizing Tau strains in a biological sample are disclosed. The methods include obtaining a sample including Tau species (e.g., including but not limited to Tau monomers and aggregated Tau species), subjecting the sample to serial amplification according to the serial amplification method disclosed herein, and evaluating biochemical, kinetic, or morphological properties of the amplified Tau fibrils. The properties are used to differentiate between Tau strains (conformers) or Tauopathy subtypes or from other neurodegenerative conditions with tau pathology such as Parkinson's disease, Huntington's disease, and others.

In some embodiments, kits for amplifying pathological Tau filaments to test compounds for use in Positron Emission Tomography (PET), but not limited to PET, are disclosed, including instructions for performing the serial amplification method disclosed herein.

In some embodiments, kits for detecting Tau filaments using PET compounds or other assays are disclosed, including instructions for performing the serial amplification method disclosed herein.

In some embodiments, the biological sample is cerebrospinal fluid, brain homogenate, cortical biopsy, spinal tissue biopsy, blood-derived exosomes, or postmortem tissue.

In some embodiments, strain differentiation is based on differences in ThT kinetics, sedimentation profiles, protease sensitivity patterns, or fibril morphology.

In some embodiments, the method identifies one or more Tauopathies selected from Alzheimer's disease, Pick's disease, PSP, CBD, AGD, CTE, GGT, MAPT mutation-associated frontotemporal dementias, or mixed Tau pathology.

In various embodiments, methods for identifying compounds that modulate Tau fibril propagation are disclosed. The methods include performing serial amplification of Tau in the presence of a test compound and comparing Tau amplification kinetics, fibril morphology, or propagation characteristics to a control reaction. A compound is identified as a modulator of Tau propagation based on changes in amplification efficiency or fibril properties.

In some embodiments, the test compound is selected from peptides, antibodies, small molecules, nucleic acids, metabolites, or post-translational modification inhibitors.

In some embodiments, a screening assay is conducted where peptides, antibodies, small molecules, nucleic acids, metabolites, or post-translational modification inhibitors may bind to monomers or the Tau filaments or fibrils in given assay conditions.

In some embodiments, modulation of amplification is quantified by changes in lag time, elongation slope, fibril mass, protease-resistance profile, or fibril ultrastructure.

In some embodiments, the assay is conducted in high-throughput or automated multiwell format.

In various embodiments, a composition including Tau fibrils produced through at least one round of serial amplification according to the serial amplification method disclosed herein is provided.

In some embodiments, the fibrils of the composition include a morphology characteristic of a specific Tauopathy-associated fold.

In some embodiments, the fibrils of the composition differ in seeding potency or cellular propagation efficiency relative to the parental seed.

In various embodiments, the serial amplification method disclosed herein is used for studying mechanisms of Tau strain replication, evolution, or structural templating.

In various embodiments, the multimodal analytical platform disclosed herein is used for clinical Tau strain stratification, research-grade structural studies, or drug discovery.

In various embodiments, serially amplified Tau fibrils are used for cryo-EM structural determination or proteomic mapping.

In some embodiments, Tau filaments may undergo structural divergence while retaining filamentous morphology and seed-dependent propagation capability.

Various embodiments include methods for serial amplification of Tau filaments, including: (a) contacting monomeric Tau protein with Tau seed material under conditions permissive for seed-dependent fibril elongation to produce amplified Tau filaments; (b) processing at least a portion of the amplified Tau filaments to generate Tau seed material for further amplification; and (c) subjecting the generated Tau seed material to one or more additional amplification steps by repeating steps (a) and (b), thereby producing serially amplified Tau filaments, where the monomeric Tau includes one or more Tau isoforms, variants, fragments, or modified forms, including 3R Tau, 4R Tau, 0N Tau, 1N Tau, or 2N Tau isoforms, truncated Tau fragments, recombinant Tau constructs, post-translationally modified Tau, disease-associated Tau variants, or any combinations thereof.

In various aspects, the methods can include where the Tau fibril seed material is derived from a Tauopathy.

In some aspects, the methods can include where the monomeric Tau includes 3R Tau isoforms, 4R Tau isoforms, or combinations thereof.

In various aspects, the methods can include where the serial amplification includes between 2 and 50 amplification cycles, each cycle diluting the Tau seed material such that the serially amplified Tau filaments are substantially free of non-Tau components originating from a biological source of the Tau fibril seed material.

In some aspects, the methods can include amplification that is performed under shaking, agitation, quiescent incubation, sonication, mechanical cycling, temperature cycling, or RT-QuIC-like conditions in the absence of added negatively charged co-factors that promote Tau aggregation.

In various aspects, the methods can include where the serially amplified Tau filaments preserve at least one structural or functional feature of the Tau fibril seed material.

In various aspects, the methods can include where the serially amplified Tau filaments exhibit structural divergence relative to the Tau fibril seed material.

In various aspects, the methods can include characterizing the amplified Tau filaments using one or more techniques selected from fluorescence kinetics, biochemical sedimentation, protease digestion profiling, mass spectrometry, electron microscopy, atomic force microscopy, or cell-based seeding assays where the characterization demonstrates substantial conversion of Tau monomers into filamentous assemblies.

In some aspects, the methods can include amplification that is conducted in a microplate, tube, microfluidic device, continuous-flow system, or automated amplification platform.

In various aspects, the methods can include where the monomeric Tau is phosphorylated, acetylated, truncated, recombinant, or chemically modified.

Various embodiments include methods for identifying a compound that modulates Tau assembly propagation, including: (a) contacting monomeric Tau protein with Tau seed material under conditions permissive for seed-dependent Tau assembly growth to produce amplified Tau assemblies, where the monomeric Tau includes one or more Tau isoforms, variants, fragments, or modified forms; (b) processing at least a portion of the amplified Tau assemblies to generate Tau seed material for further amplification; (c) repeating steps (a) and (b) one or more times to produce serially amplified Tau assemblies; (d) performing the serial amplification of steps (a)-(c) in the presence of a test compound; (e) performing the serial amplification of steps (a)-(c) in the absence of the test compound as a control; and (f) comparing one or more propagation characteristics of the Tau assemblies produced in steps (d) and (e), where the test compound is identified as modulating Tau assembly propagation based on a difference in at least one propagation characteristic selected from amplification kinetics, assembly morphology, seeding activity, biochemical properties, or cellular propagation behavior.

In various aspects, the methods can include where the test compound is selected from small molecules, peptides, antibodies, nucleic acids, metabolites, or post-translational modification inhibitors.

In some aspects, the methods can include where modulation of Tau assembly propagation is quantified by changes in lag time, elongation rate, fibril mass, protease resistance, or fibril ultrastructure.

In various aspects, the methods can include where the method is conducted in a high-throughput or automated multiwell screening format.

In various aspects, the methods can include where the test compound modulates Tau assembly propagation by interacting with Tau monomers, Tau assemblies, or Tau assembly ends.

Various embodiments can include methods for serial amplification of Tau assemblies, including: (a) contacting monomeric Tau protein with Tau seed material obtained from a biological sample under conditions permissive for seed-dependent Tau assembly growth to produce amplified Tau assemblies; (b) processing at least a portion of the amplified Tau assemblies to generate Tau seed material for further amplification; and (c) subjecting the generated Tau seed material to one or more additional amplification steps by repeating steps (a) and (b), thereby producing serially amplified Tau assemblies, where the Tau assemblies include aggregated Tau species capable of templated propagation, and where the monomeric Tau includes one or more Tau isoforms, variants, fragments, or modified forms.

In various aspects, the methods can include where the Tau seed material is obtained by homogenization and centrifugation of the biological sample.

In some aspects, the methods can include where the biological sample includes cerebrospinal fluid, brain homogenate, blood-derived extracellular vesicles, or postmortem tissue.

In various aspects, the methods can include where the serially amplified Tau assemblies are used to differentiate Tau conformational strains or Tauopathy subtypes.

In various aspects, the methods can include where the serially amplified Tau assemblies are used for structural, biochemical, or functional analysis.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure. As used in the specification and in the claims, the singular form of ‘a,’ ‘an,’ and ‘the’ include plural referents unless the context clearly dictates otherwise.

As discussed herein, it would be desirable in the medical industry for improved methods and associated systems for generation and amplification of full-length Tau filaments, particularly useful in the search for diagnostic and therapeutic approaches for neurodegenerative disorders.

In various aspects, Tau filaments may be a defining characteristic of Alzheimer's disease (AD) and numerous other neurodegenerative disorders. In some aspects, the deposition of Tau protein into aggregates involves templated recruitment of Tau monomers onto the filament ends, via their microtubule-binding repeats. This structural conversion may be central to the propagation of Tau pathology, yet its molecular mechanisms are still poorly understood. Specifically, it is unclear whether co-factors are required for templated growth. To gain insights into this process, the serial amplification of pathological filaments from AD, Pick's disease (PiD), and progressive supranuclear palsy (PSP) was probed in various embodiments. These filaments are made from different compositions of three- and four repeat- (3R and 4R) Tau. Illustrative results observe that AD Tau filaments recruit full-length 3R and 4R Tau in the absence of co-factors at low salt and that these filaments can be independently amplified over multiple generations. Similarly, PiD Tau and PSP Tau filaments can also be serially amplified. The generated filaments can retain the cross-seeding properties of the pathological seeds; PSP filaments recruit only 4R Tau, PiD filaments recruit only 3R Tau, and AD filaments recruit both. The findings disclosed herein advantageously demonstrate that co-factors are not needed for templated growth and suggest that the conformational state of the monomer ensemble determines whether fibrils propagate.

Various embodiments described herein relate to methods and systems for the serial, seed-dependent amplification of Tau assemblies, including Tau filaments and fibrils, using pathological Tau material as an initial template and monomeric Tau protein as a substrate. The disclosed approaches can enable controlled propagation of disease-associated Tau assemblies under co-factor-free conditions, thereby facilitating the generation of structurally defined Tau assemblies suitable for biochemical, structural, and functional analyses, including drug-screening applications.

Some embodiments described herein demonstrate the generation of Tau filaments using pathological Tau filaments as Tau seed material and recombinant Tau protein for fibril elongation. The Tau filaments may be generated from brain extracts from tauopathy patients, such as patients suffering from Alzheimer's disease, progressive supranuclear palsy (PSP), Pick's disease (PiD), and others. The filaments generated from different tauopathies can result in Tau filaments with different molecular structures (conformations) from each other.

In some embodiments, Tau assemblies may be generated by contacting monomeric Tau protein with Tau seed material derived from a tauopathy-associated biological source, such as brain tissue from individuals affected by Alzheimer's disease (AD), progressive supranuclear palsy (PSP), Pick's disease (PiD), corticobasal degeneration, or other tauopathies. Distinct tauopathies may give rise to Tau assemblies having different molecular conformations, and the disclosed methods enable propagation of such disease-specific features.

Some aspects of the embodiments described herein include that they do not require negatively charged co-factors, which are known to interfere with drug screening and other relevant assays. Prior techniques for generating Tau filaments depend on the presence of co-factors. For example, prior technique protocols require 30-fold higher protein concentrations (such as 300 uM and more) and do not use pathological fibrils as templates. The prior technique protocols furthermore use highly truncated versions of Tau (>75% of the amino acid sequence is removed).

Unlike certain existing approaches that rely on negatively charged co-factors or highly truncated Tau constructs, embodiments described herein enable seed-dependent elongation of Tau assemblies in the absence of added aggregation-promoting co-factors. In some embodiments, full-length Tau isoforms, including three-repeat (3R) and four-repeat (4R) Tau, as well as N-terminal splice variants (0N, 1N, 2N), truncated fragments, disease-associated variants, post-translationally modified Tau, recombinant Tau constructs, or combinations thereof, may be employed as monomeric substrates.

In various embodiments, Tau monomers elongate onto pre-existing Tau seeds in a template-assisted manner, thereby substantially preserving one or more structural or functional features of the original seed material. In other embodiments, amplification under defined conditions may result in assemblies that diverge structurally from the original seed material while retaining templated propagation capability.

In some embodiments, and without being bound by theory, the ability of monomeric Tau to participate in seed-dependent elongation may depend on the conformational state of the Tau monomer. Although Tau is intrinsically disordered, monomeric Tau may adopt transient conformations that reduce accessibility of aggregation-prone motifs. Under conditions permissive for amplification, including but not limited to reduced ionic strength, defined buffer composition, agitation, mutation, truncation, or post-translational modification, monomeric Tau may adopt a more aggregation-competent conformation that facilitates recruitment onto pre-existing Tau assemblies. Such conditions may promote efficient templated propagation in the absence of negatively charged co-factors.

In various embodiments described herein, one or more structural features of the original seed assemblies are preserved as Tau monomers elongate onto the seeds in a template-assisted manner. In some embodiments, serial amplification under selected conditions may yield amplified Tau assemblies that differ in at least one structural feature relative to the original seed material while retaining templated propagation capability. In some embodiments, full-length three-repeat (3R) and four-repeat (4R) Tau isoforms, truncated versions, and variants may be used. In this way, once generated, the filaments can be propagated indefinitely through repeated cycles of seeding and growth.

Another disadvantage of prior techniques is that they result in conformationally heterogeneous fibrils. High protein concentrations, structural heterogeneity of the resulting filaments, and overall dependence on co-factors are characteristic features of current protocols that limit the discovery of novel drugs/molecules interfering with the elongation of Tau filaments. That is, according to embodiments described herein, Tau filament-containing brain extracts (or purified filament from brain) are only required for the initial amplification, and large amounts of brain extracts are not required. Thereafter, virtually any quantity of disease-specific Tau filaments may be generated in vitro using mixtures of recombinant Tau proteins and Tau seed material.

In embodiments, the described methods include multistep amplification of pathological filaments. As used herein, “Tau assemblies” may include Tau filaments, fibrils, and other Tau species (e.g., including but not limited to Tau monomers and aggregated Tau species) capable of templated propagation. The resulting filaments can be used for the screening of potential drug candidates that inhibit fibril elongation, as it is recognized that fibril elongation is central to the propagation and spreading of pathological Tau filaments throughout the human brain. In some embodiments, screening includes performing serial amplification in the presence of a test compound and, in parallel, performing serial amplification in the absence of the test compound as a control, and comparing one or more propagation characteristics including amplification kinetics, lag time, elongation rate, fibril mass, morphology, protease resistance, seeding activity, or cellular propagation behavior. The filaments may also be used for applications that probe the binding of diverse biomolecules and for applications that study basic mechanisms of Tau aggregation. As used herein, functional analysis includes assessing biological or cellular effects of Tau assemblies, including seeding activity, propagation efficiency, cytotoxicity, or aggregation behavior in cell-based or other biological assay systems. In some embodiments, a test compound modulates Tau assembly propagation by interacting with monomeric Tau, aggregated Tau assemblies, or termini of Tau assemblies, thereby altering one or more propagation characteristics. In some embodiments, test compounds modulate Tau propagation by binding to elongation-competent ends of Tau assemblies, lateral surfaces of Tau filaments, or soluble monomeric Tau, thereby inhibiting, enhancing, or altering assembly growth dynamics. Accordingly, serially amplified Tau assemblies produced by the methods described herein are suitable for functional analysis, including evaluation of seeding competence, propagation efficiency, cytotoxicity, or biological activity in cellular or other biological systems. Propagation includes fibril/filament elongation and amplification of assemblies.

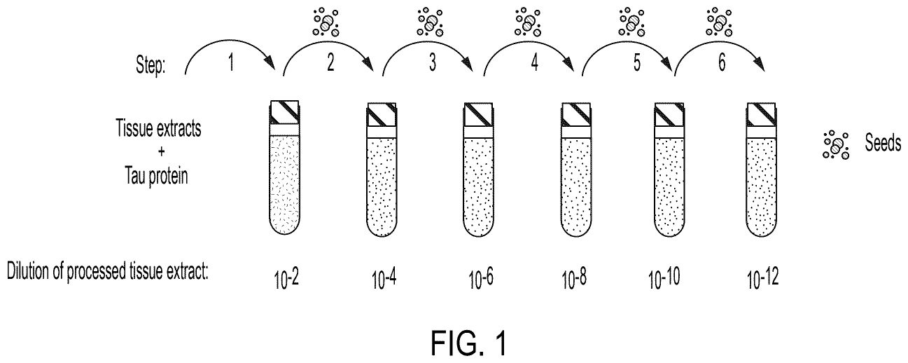

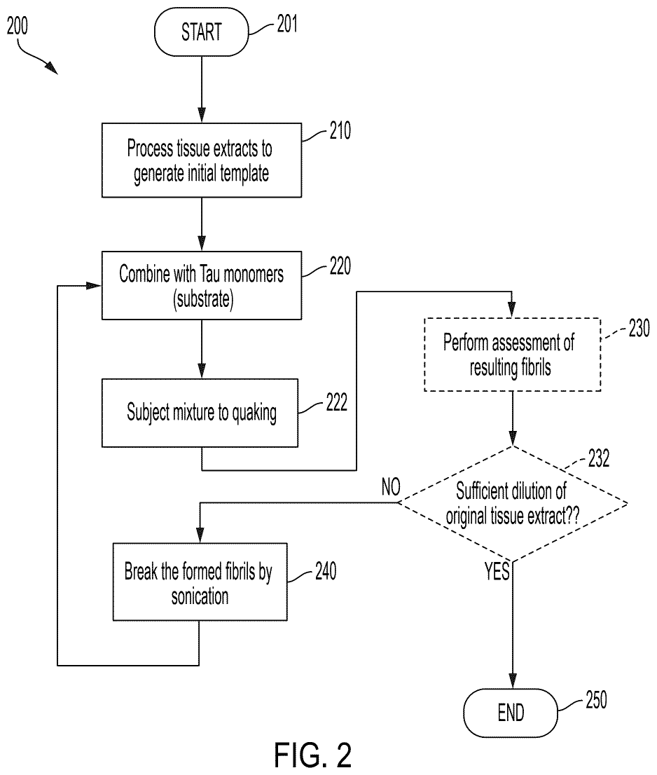

FIGS. 1 and 2 illustrate an exemplary multistep amplification process. In some aspects, a single amplification cycle includes: mixing Tau seed material with monomeric Tau protein to form a reaction mixture; incubating the mixture under conditions permissive for seed-dependent assembly growth, which may include shaking, quaking, quiescent incubation, or combinations thereof, and processing at least a portion of the resulting Tau assemblies to generate new seed material, for example by sonication or other mechanical disruption. The processing may promote fibril breakage. The newly generated seed material may then be combined with additional monomeric Tau protein to initiate a subsequent amplification cycle. These steps may be repeated multiple times, including between 2 and 50 cycles or more, thereby producing serially amplified Tau assemblies while progressively diluting non-Tau components originating from the initial biological source. In some embodiments, each amplification cycle dilutes the original biological material by at least 10-fold, 100-fold, or more, such that after multiple cycles the resulting Tau assemblies are substantially free of non-Tau contaminants.

Formation and propagation of Tau assemblies may be monitored in real time or post-amplification using one or more analytical techniques. Exemplary techniques include fluorescence-based assays (e.g., Thioflavin T kinetics), biochemical sedimentation, protease digestion profiling, mass spectrometry, electron microscopy, atomic force microscopy, or cell-based seeding assays. In some embodiments, assessment results may be used to determine whether additional amplification cycles are performed or whether the process is terminated after achieving a desired level of dilution or assembly yield.

FIG. 1 graphically illustrates steps involved in a multistep Tau amplification assay from a tissue extract, in accordance with various embodiments disclosed herein. As shown in FIG. 1, a sample of tissue extract is processed and combined with Tau protein monomers. In the illustrated example, the reactions in each step are diluted in such a way that in every generation the reaction is diluted 100-fold (e.g., after step 1 the processed tissue extract is diluted to 10⁻², to 10⁻⁴ after step 2, and so on), and Tau fibrils are amplified by addition of new Tau monomers.

In an exemplary experimental workflow, in accordance with FIG. 1, post-mortem brain tissue from disease and healthy individuals was processed and incubated with recombinantly produced Tau proteins and a fluorescent Thioflavin T (ThT) dye. A Real-Time Quaking-Induced Conversion (RT-QuIC) assay is used to amplify Tau assemblies from proteopathic seeds present in processed disease biospecimens (e.g., disease brain biospecimens) to form elongated fibrillar assemblies (step 1). RT-QuIC is an exemplary approach that combines intermittent agitation (e.g., quaking or shaking) with incubation to promote seed-dependent elongation, and assay parameters may be adjusted depending on the seed material, monomeric Tau substrate, and desired output. The formation of fibrils can be determined in real time, for example, by measuring ThT fluorescence kinetics or after the experiments, such as by sedimentation assay or visualization by electron microscopy.

In various embodiments, the resulting fibrils are sonicated for 30 seconds for breakage, combined with new Tau monomers and subjected to another round of quaking (step 2). In various aspects, whereas quaking or shaking may be considered to be a gentle method for breaking fibrils into seeds with lengths on the order of hundreds of nanometers, sonication may be regarded as a more harsh method for generating short fibril seeds (e.g., about 50 nanometers (nm)). The actual length of the resulting fibril seeds may generally be a factor of the shaking speed and the type of fibril used. The steps (fibril sonication, mixing with monomers, and quaking) can be repeated indefinitely leading to serial (multistep) amplification of Tau filaments. That is, while the original brain material is diluted, the resulting fibril seeds can become the template for additional fibril growth such that the fibril growth process may be repeated indefinitely.

In embodiments, the multistep Tau amplification assay begins by first enriching Tau filaments from a selected tauopathy case (such as Alzheimer's disease (AD), progressive supranuclear palsy (PSP), Pick's disease (PiD), and others) through homogenization and centrifugation. These samples are then sonicated, mixed with recombinant Tau protein in the absence of co-factors, and incubated in a microplate reader with intermittent shaking. Filament formation is monitored by Thioflavin T (ThT) fluorescence, sedimentation, and electron microscopic imaging. As observed by these monitoring approaches, in various embodiments, the protocol allows for the substantial, complete, or near-complete conversion of Tau monomers into filaments. The generated filaments are sonicated and serve as seeds for the next generation of fibrils. This process can be repeated indefinitely, and the generated fibrils may be siphoned off at any stage. As used herein, removal or “siphoning off” of Tau assemblies at any stage refers to removal at any amplification generation or cycle, while allowing remaining material to continue through subsequent amplification rounds. In some embodiments, the methods and systems described herein enable substantial or near-complete conversion of full-length Tau monomers into filamentous assemblies under co-factor-free conditions. In some embodiments, substantial or near-complete conversion corresponds to conversion of at least 80%, at least 90%, or at least 95% of monomeric Tau into filamentous assemblies as assessed by sedimentation or biochemical fractionation.

FIG. 2 is a process flow chart illustrating a multistep Tau amplification assay from a tissue extract, in embodiments. Generally, the multistep amplification process of the present disclosure includes: 1) mixing the template fibril seeds with monomers to generate a mixture; 2) incubating and quaking the mixture to allow monomers to efficiently grow onto the seeds (a certain amount of quaking-assisted breakage of the fibrils may help speed up the growth process); and 3) sonicating the resulting fibrils in the processed sample to generate new fibril seeds. Steps 1)-3) are repeated with the generated new fibril seeds and new monomers. That is, the new fibrils/seeds become templates, while the monomers function as the substrate that is converted into a new conformation. In other words, a single step (e.g., as shown in FIG. 1) involves mixing fibril seeds with Tau monomers, subjecting the mixture to quaking (shaking), then sonicating the resulting fibrils to generate new fibril seeds.

In some embodiments, “processing” or “mechanical disruption” of Tau assemblies to generate Tau seed material refers to generating seed-competent fragments that retain the ability to template recruitment of monomeric Tau into an assembly conformation. Seed-competent fragments may have a distribution of lengths and may be generated by agitation, quaking, shear, or sonication as described herein. In some embodiments, the degree of fragmentation is selected to balance (i) generating sufficient numbers of growth-competent ends for efficient amplification and (ii) preserving one or more structural features of the parental assemblies.

In various aspects, Tau fibril seed material may have a minimum length on the order of about 1 nm. In various embodiments, Tau fibril seeds generated by mechanical disruption, such as sonication, exhibit characteristic lengths on the order of tens of nanometers, for example about 40-50 nm, as observed by electron microscopy. In some embodiments, further sonication does not substantially reduce seed length below this range. In various embodiments, Tau fibril seed material may include longer fibril fragments; however, as seed length increases, the number of growth-competent ends per unit mass decreases, which may reduce seeding efficiency. Without being bound by theory, seed-dependent Tau assembly growth occurs primarily at fibril ends, such that seed length and fragmentation state influence the efficiency of monomer recruitment and filament elongation.

In some aspects, the fibrils may be tip-sonicated for about 30 seconds (s) on ice. For example, the sonicator tip may lowered into the tube and the power switched on with power settings that may depend on volume, instrument, and type of tip. In some embodiments, the duration of mechanical disruption, such as sonication, may be increased to promote additional fragmentation of Tau assemblies. In such embodiments, care may be taken to limit excessive heating of the sample. For example, mechanical disruption may be performed in an intermittent manner, such as applying sonication for a defined interval (e.g., about 10-60 seconds), followed by a cooling period (e.g., incubation on ice), and optionally repeating the cycle one or more times.

In various embodiments, after 30s the fibrils may have reached their minimal length (e.g., about 40-50 nm) and after that, the Tau assemblies may become resistant to further mechanical fracture under the applied processing conditions. For example, the Tau assemblies may become resistant to further mechanical fracture because the fibrils become too short. In some embodiments, bath sonication may be used to fracture the fibrils. In some embodiments, when lower-power mechanical disruption is employed, longer processing durations may be used, including durations on the order of minutes to tens of minutes. In some embodiments, fibrils treated by bath sonication are much longer than the ones treated by tip-sonication. In various aspects, short fibrils are desired since this increases the number of ends per mass and the assay becomes more efficient. In some aspects, relatively low amounts of Tau fibril seed material may be transferred from one amplification cycle to a subsequent cycle. In such embodiments, the amount of seed material may be expressed in terms of Tau monomer equivalents (e.g., total Tau protein concentration) rather than the number of individual seed particles. For example, Tau fibril seed material may be diluted at a ratio of about 1:100 between amplification cycles.

In various embodiments, the effective number of seed-competent fibril ends depends on the degree of fragmentation of the Tau assemblies. If mechanical disruption is less efficient and results in longer fibril fragments, the number of growth-competent ends may be reduced. In such cases, a higher Tau protein concentration and/or a higher input of Tau fibril seed material may be employed to achieve a comparable number of effective seeds and maintain amplification efficiency.

In some embodiments, various advantages of sonication include generation of relatively shorter Tau fibril seeds compared to agitation-based fragmentation. In some aspects, however, sonication may be less readily compatible with real-time optical readouts during an amplification reaction. For example, RT-QuIC assays employ intermittent shaking and enable real-time monitoring of Tau assembly growth (e.g., using fluorescence-based detection).

In various embodiments, upon completion of an amplification cycle (generation) monitored using RT-QuIC or another assay, the resulting Tau fibrils may be removed from the detection format and subjected to mechanical disruption, such as sonication, to generate seed material for a subsequent amplification cycle. In some embodiments, the material may be diluted between cycles, and processing can be performed outside the optical detection system. In some embodiments it is advantageous to employ sonication at this stage to generate shorter fibril fragments than those produced by shaking alone, thereby increasing the number of seed-competent ends for subsequent amplification rounds. In various embodiments, microfluidic flow may be used to shear the fibrils.

In various aspects, initiation of an amplification reaction includes providing a sufficient quantity of growth-competent Tau fibril seeds, for example by mechanical fragmentation such as sonication. Once monomeric Tau is introduced and assembly growth is underway, monitoring of the amplification reaction may be performed. In some embodiments, a RT-QuIC assay is used to enable real-time monitoring of Tau assembly growth, for example via an optical or fluorescence-based readout. In such embodiments, agitation or shaking applied during the amplification reaction may be sufficient to fracture elongating fibrils in a controlled manner, thereby gradually (e.g., continuously but gently) increasing the concentration of growth-competent seed ends as the reaction progresses. In alternative embodiments, the amplification reaction may be performed without real-time optical monitoring, for example by incubation with agitation in a shaker or similar device, and the reaction outcome may be evaluated after completion using post hoc analytical techniques such as biochemical sedimentation, gel electrophoresis, or electron microscopy.

In some aspects, one or more structural features of the Tau fibrils are preserved during serial amplification, while the fibrils are reduced in length through mechanical processing. In such embodiments, fibril fragmentation is performed between amplification cycles, for example by sonication, to generate a larger number of growth-competent seed fragments. Sonication may produce shorter fibril fragments, for example on the order of tens of nanometers, whereas agitation or shaking alone typically produces longer fibrils due to lower applied mechanical energy. In some embodiments, fragmentation by agitation results in fibrils that are substantially longer than those produced by sonication. Although precise length distributions may vary and may not be necessarily quantified in all embodiments, sonication is selected in certain implementations to increase the number of seed-competent ends for subsequent amplification cycles. In some embodiments, care is taken to limit excessive heating of the sample during sonication, as elevated temperature may reduce seeding activity or compromise structural integrity of Tau assemblies. Accordingly, sonication may be performed under controlled conditions, such as intermittent processing and/or cooling, to maintain seeding competence.

This process of mixing, quaking, and sonicating may be repeated indefinitely. In some embodiments, the process is performed for a predetermined number of amplification cycles (e.g., between 2 and 50 cycles), with each cycle diluting the original seed source such that the resulting Tau assemblies are substantially free of non-Tau components present in the initial biological material. As a result, the originally processed tissue extract (along with human disease fibrils) may be diluted indefinitely, while synthetic fibrils with the same properties as the disease fibrils are simultaneously generated. Every time the fibril seeds are mixed with recombinant monomers, the original brain tissue material is diluted further such that the resulting sample may be considered to be pure and free of brain contaminants.

As used herein, “substantially free of non-Tau components” refers to a reduction of non-Tau material originating from the biological source to a level that does not materially interfere with intended downstream analyses or applications, including biochemical characterization, structural studies, or screening assays. In some embodiments, substantial freedom from non-Tau components is evidenced by one or more of: (i) progressive dilution of the initial biological source by at least 10-fold or at least 100-fold per amplification cycle over multiple cycles; (ii) a predominance of Tau signal in sedimented filament fractions relative to detectable non-Tau proteins by gel-based analysis; and/or (iii) the ability to reproducibly propagate Tau assemblies using recombinant monomeric Tau as substrate without requiring replenishment of the original biological material.

This purification result provides an additional advantage for the embodiments described herein over the existing art. In particular, it is recognized herein that the only currently available method to obtain pathological fibrils is to purify the fibrils from brain extract. This existing process to obtain pathological fibrils from brain extract is performed through multiple steps of complex immunopurification protocols or other means. Unfortunately, the large amounts of brain tissue required for the existing process may not be available or be difficult to obtain, and processing of the brain tissue to obtain pure pathological fibrils may be difficult. In contrast, the embodiments described herein can advantageously achieve large quantities of assemblies (including fibrils) from only a small amount of brain tissue extract. In various aspects, the resulting pathological fibrils may be considered pure after multiple dilutions.

As shown in FIG. 2, a process 200 for a multistep Tau amplification assay begins with a start step 201 and proceeds to a step 210 to process tissue extracts to generate an initial template. The tissue extracts may be obtained, for example, as post-mortem brain tissue from diseased or healthy individuals. In other embodiments, the biological sample may include cerebrospinal fluid, brain homogenate, blood-derived extracellular vesicles, or other biospecimens containing Tau seed material. For instance, the tissue extract may contain Tau fibrils from a tauopathy patient.

In some embodiments, when the biological sample includes cerebrospinal fluid (CSF), Tau seed material may be obtained by concentrating the CSF and/or enriching aggregated Tau species prior to initiating serial amplification. By way of example, CSF may be clarified to remove cellular debris, optionally subjected to concentration (for example by centrifugal filtration), and optionally fractionated to enrich aggregated or high-molecular-weight Tau species. The resulting concentrate or enriched fraction may be used as Tau seed material in the contacting step with monomeric Tau protein.

In some embodiments, Tau seed material may be enriched or isolated using immunoprecipitation-based techniques. For example, aggregated Tau species may be pulled using an antibody that binds Tau assemblies, optionally subjected to one or more washing steps, and subsequently mechanically processed (e.g., by shearing or sonication) to generate seed-competent fragments. The resulting Tau seed material may then be combined with Tau monomers to initiate serial amplification. In various embodiments, immunoprecipitation may provide an optional enrichment step.

In some embodiments, when the biological sample includes blood-derived extracellular vesicles (including exosomes), Tau seed material may be obtained by isolating extracellular vesicles and releasing vesicle-associated Tau assemblies. For example, vesicles may be isolated from blood, plasma, or serum using precipitation, size-based separation, ultracentrifugation, immunocapture, or combinations thereof. Vesicles may then be lysed or otherwise processed to release vesicle-associated Tau species, including aggregated Tau, and the resulting preparation may be used as Tau seed material for serial amplification.

In some embodiments, when the biological sample includes a biopsy, homogenate, or postmortem tissue, Tau seed material may be enriched by homogenization in a buffered solution followed by one or more centrifugation steps to remove insoluble debris and/or to enrich filamentous material. In some embodiments, the enriched material is optionally subjected to mechanical processing, including sonication, to generate seed-competent fragments prior to contacting with monomeric Tau.

In some embodiments, enrichment or fractionation of Tau seed material from a biological sample is performed in a manner that preserves seeding activity and reduces non-Tau components. In some embodiments, the serial amplification process itself provides progressive dilution of non-Tau components originating from the biological sample across amplification cycles, thereby yielding Tau assemblies that are substantially free of non-Tau contaminants after multiple rounds of amplification.

Process 200 then proceeds to a step 220 to combine the tissue extract with Tau monomers, which serve as the substrate for subsequent reactions. In embodiments, step 220 may also include incubating the tissue extract with recombinantly produced Tau proteins and a fluorescent dye (e.g., Thioflavin T (ThT) dye) for use in assessments of the amplification results. Step 220 may additionally include setting up the mixture for the amplification reaction process in an appropriate assay format, such as in a 96 well plate.

The multistep Tau amplification assay process of FIG. 2 proceeds to a step 222 to subject the mixture to quaking or shaking. The quaking may be performed, for example, using a RT-QuIC assay or performed without a real-time readout (e.g., an amount of Tau assembly formation may be assessed after completion of the reaction using one or more post-reaction analytical techniques, such as sedimentation analysis, gel electrophoresis, immunodetection, or electron microscopy). In embodiments, the processing of steps 220 and 222 enable the Tau proteins from proteopathic seeds present in the processed disease brain biospecimens to be amplified to long fibrils.

In some embodiments, in place of shaking with the RT-QuIC assay, the amplification of the present disclosure may be achieved by other mechanical means such as repeated cycles of sonication. However, in some aspects, the power output of, for example, bath sonicators may be more difficult to control compared to the quaking provided by the RT-QuIC assay, which is generally more readily controllable and reproducible. For instance, sonication (such as tip sonication) may be more appropriate for generation of large amounts of fibril seeds for a subsequent step in the amplification process.

Optionally, the amplification and formation of fibrils may be monitored by performing an assessment of resulting sample in a step 230 as shown in FIG. 2. For instance, formation of the Tau fibrils may be determined in real time by measuring ThT fluorescence kinetics during the amplification assay. As a further option, based on the results of assessment step 230, a determination 232 may be made whether sufficient dilution has been completed such that the generated Tau filaments are considered pure from tissue contaminants. In this case, if the answer to determination 232 is NO, then process 200 proceeds to a step 240 to break the amplified fibrils by sonication to produce new fibril seeds. The newly produced fibril seeds may then be used as templates in additional amplification steps by returning to step 220 to repeat combining step 220 and quaking step 222. If the answer to determination 232 is YES, then process 200 proceeds to an end step 250 to terminate the process.

Steps 220, 222, and 240 (and optionally 230 and 232) may be repeated to achieve serial amplification of pathological Tau elements while diluting the original tissue extracts. This cyclical process may be terminated when the desired dilution of the originally processed brain tissue has been achieved. As shown in FIG. 1, the amplification assay, assessment, and determination steps 220, 222, and 240 may be repeated to achieve the desired dilution of originally processed brain extract while the Tau fibrils are propagated to the next iteration as desired. Alternatively, the amplification process may be terminated after a preset number of iterations.

Alternatively or additionally, the resulting sample after amplification process 200 may be removed from the assay system and assessed in a subsequent step, for example, by a sedimentation assay and/or imaging (e.g., electron microscopy).

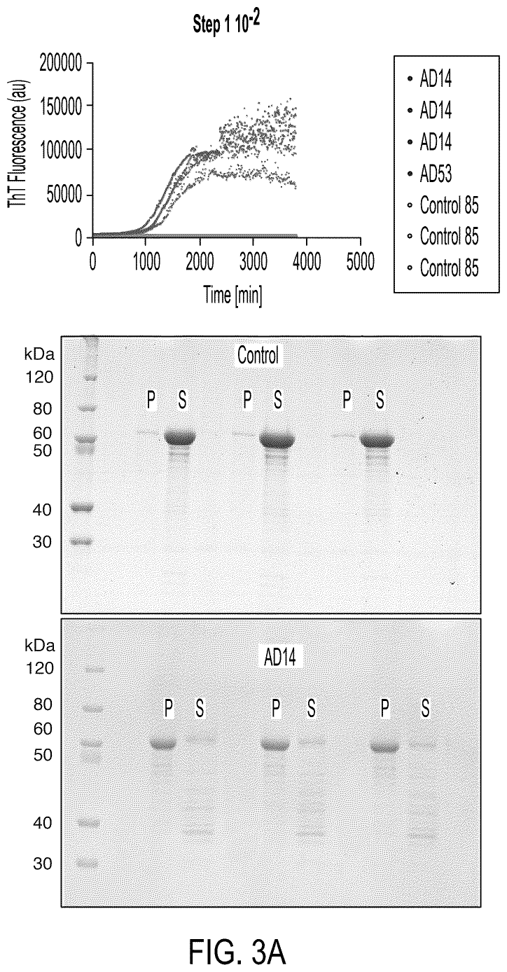

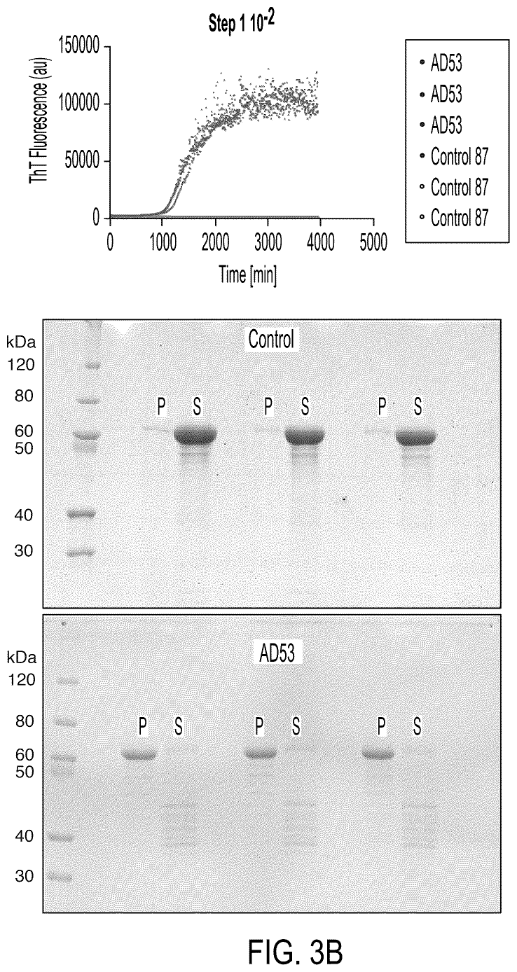

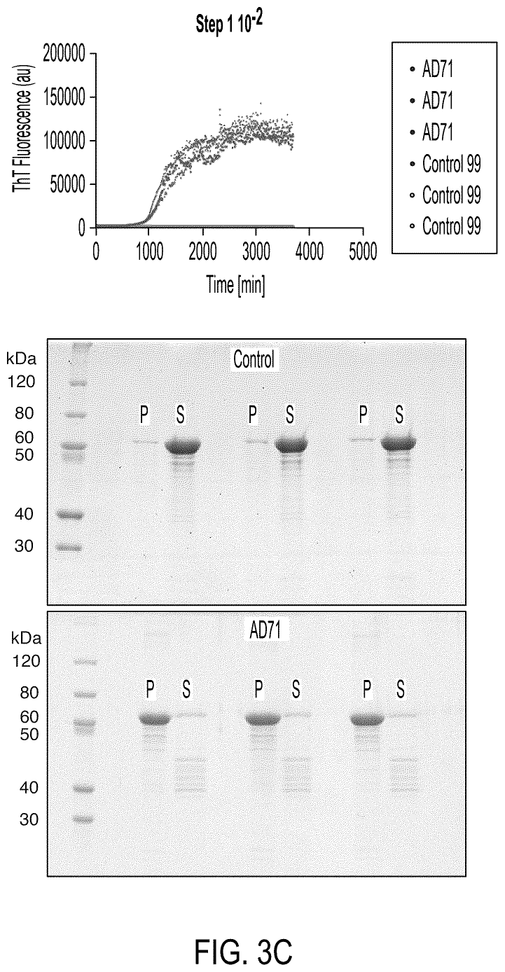

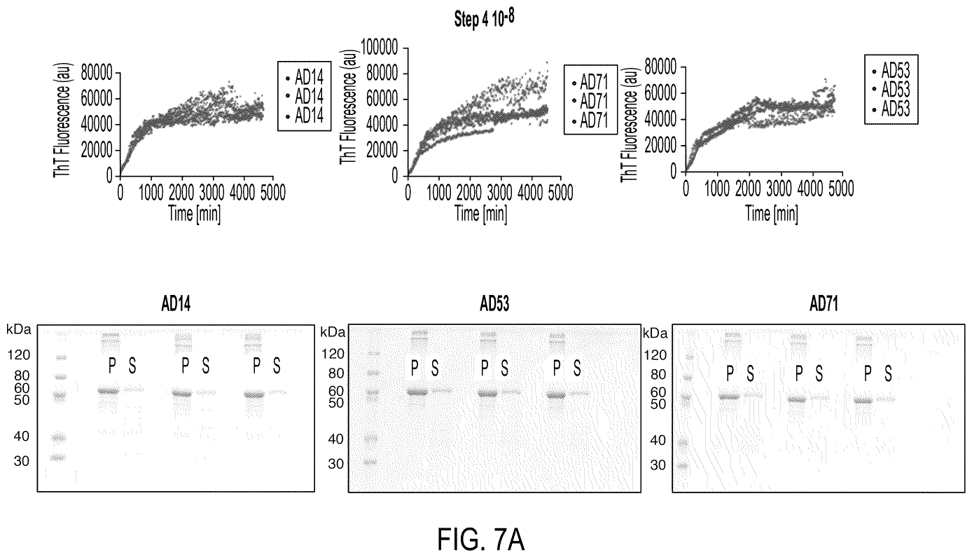

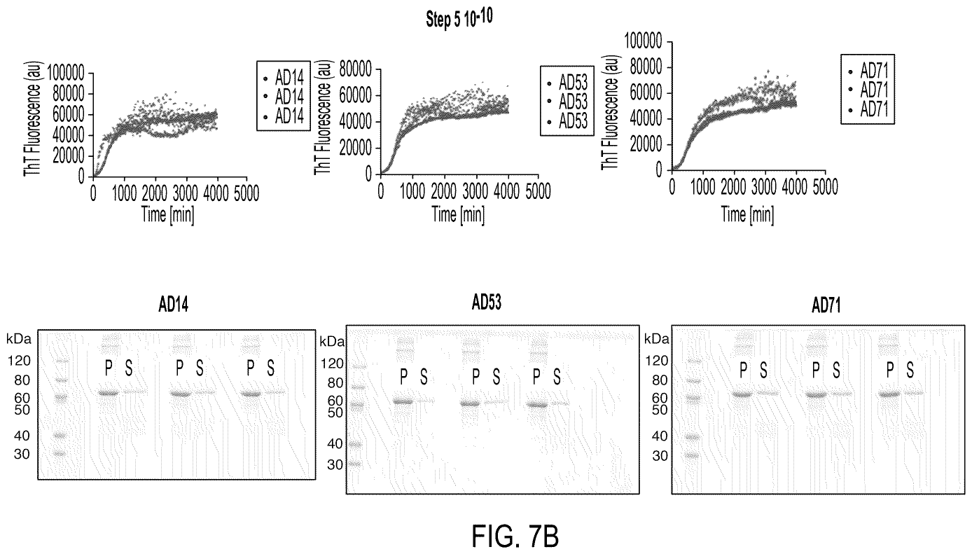

Experimental results, e.g., as illustrated in FIGS. 3A-16, demonstrate that Tau assemblies derived from different tauopathies may exhibit isoform-specific amplification behavior. For example, AD-derived seed material may support amplification of both 3R and 4R Tau isoforms, whereas PSP-derived seeds may preferentially amplify 4R Tau, and PiD-derived seeds may preferentially amplify 3R Tau. Such disease-specific characteristics may be exploited to differentiate Tau conformational strains or tauopathy subtypes. As used herein, a “Tau strain” refers to a conformationally distinct Tau assembly characterized by a reproducible molecular fold, ultrastructure, and propagation behavior, independent of Tau amino acid sequence variation.

The serially amplified Tau assemblies described herein may be used in a variety of applications, including and not limited to: screening and identifying compounds that modulate Tau propagation or assembly elongation; structural and biochemical characterization of Tau conformations; cell-based assays to evaluate seeding activity, toxicity, or propagation behavior; and fundamental studies of Tau aggregation mechanisms.

Although illustrated as discrete steps, the operations shown in FIGS. 1 and 2 may be combined, reordered, performed iteratively, or partially automated, and individual steps may be omitted or repeated depending on the desired amplification outcome.

FIGS. 3A-3C show experimental results from Step 1 of the multistep Tau amplification assay of FIGS. 1 and 2, shown to illustrate the formation of Tau fibrils from brain extract of an Alzheimer's Disease (AD) patient, in accordance with various embodiments disclosed herein. In particular, full length 4R Tau mutant monomer P301S is added to Alzheimer's disease brain extract as well as healthy control under RT-QuIC assay conditions at 37° C. Binding of Thioflavin T to Tau fibrils results in fluorescence in disease material but not control material indicating the formation of Tau fibrils from disease brain extract (FIGS. 3A-3C, scatter plots).

It is recognized herein that both full-length 3R and 4R Tau (wild-type) can be amplified, noting that AD filaments are generally composed of both 3R and 4R Tau. The multistep amplification of both types of isoforms and the ability of step 6 (as shown in FIG. 1) 4R Tau fibrils to recruit 3R tau (and vice versa) underscores that the structure may be preserved. In embodiments described herein, the amplification process is successful with both the variant Tau mutant monomer P301S and wildtype Tau monomers. Therefore, as described herein, full length 3R and 4R Tau proteins, truncated versions, and variants, including wildtype Tau monomers, may be used in the multistep amplification process in embodiments.

For other tauopathies, the multistep amplification has been shown to work differently from AD filaments. For instance, when PSP fibrils are used, it appears that only 4R Tau can be amplified. When PiD fibrils are used, only 3R Tau can be amplified. Such results may be expected as PSP is known to be a 4R tauopathy, whereas PiD is recognized as a 3R tauopathy.

The resulting proteins were sedimented and analyzed by SDS-PAGE and Coomassie staining. The control brain showed no protein in the pellet (indicated as “P”) fraction indicating that no fibrils were formed, all of the protein was found in the supernatant (indicated as “S”) indicating this protein was still monomer in all three brains (FIGS. 3A-3C, controls). The disease brain showed most of the protein in the pellet fraction as opposed to the supernatant fraction indicating that the protein was mostly in its fibrillar form (FIGS. 3A-3C, AD14, AD53, and AD71).

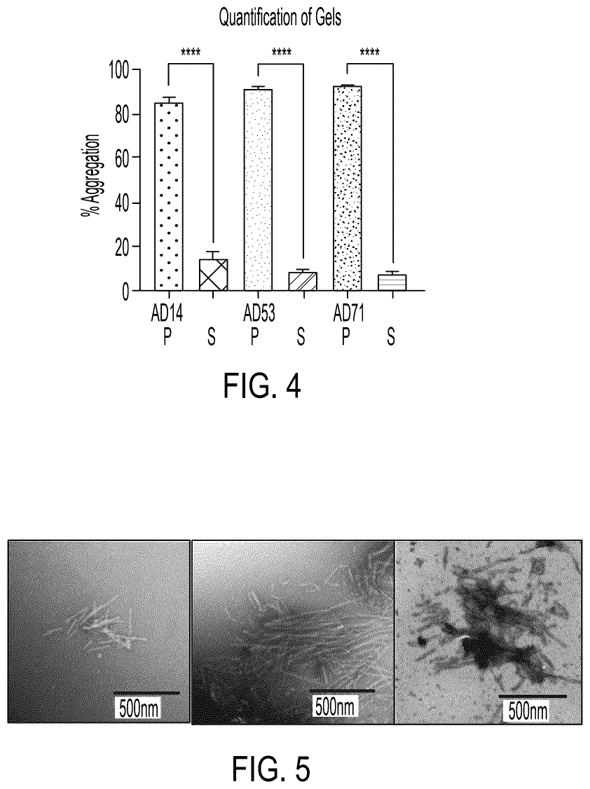

FIG. 4 shows a bar graph illustrating the quantified SDS-PAGE and Coomassie staining results of FIGS. 3A-3C. In particular, a quantification of the Alzheimer's gels and statistical comparison using one-way ANOVA test, with **** P≤0.0001. The error bars represent means±standard deviation (SD).

FIG. 5 shows transmission electron microscopy (TEM) images illustrating the formation of disease fibrils from brain tissue extract as corresponding to the results shown in FIGS. 3A-3C. As visible in FIG. 5, the TEM images show disease fibrils formed from brain material.

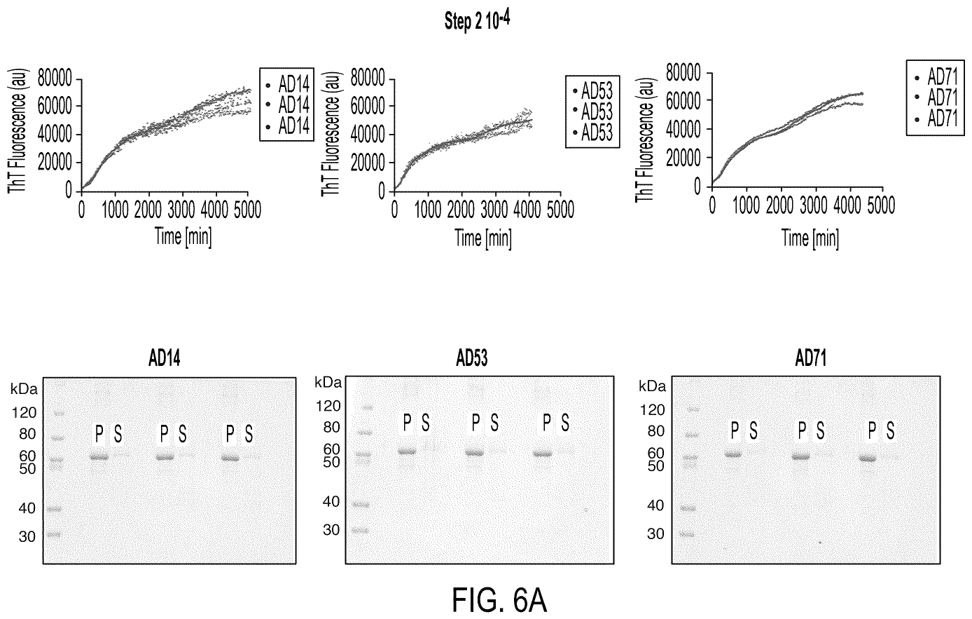

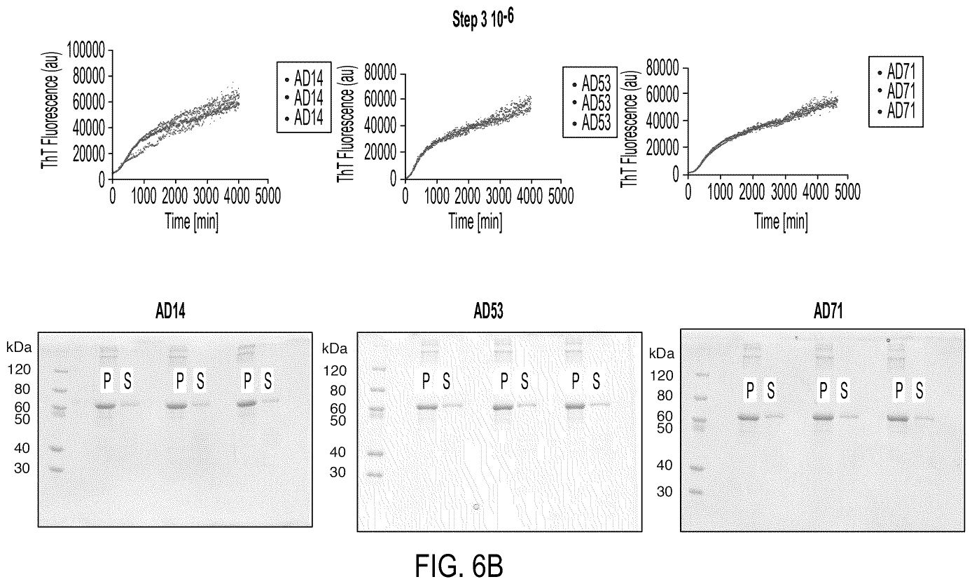

FIGS. 6A, 6B, 7A, 7B, and 8 show experimental results from Steps 2-6 of the multistep Tau amplification assay of FIGS. 1 and 2, shown here to further illustrate the formation of Tau fibrils from diseased brain extract, in accordance with various embodiments disclosed herein. In the illustrated example, full length 4R Tau mutant monomer P301S is added to brain tissue seeds generated in the process illustrated in FIGS. 1 and 2. Binding of Thioflavin T to Tau fibrils results in fluorescence in disease material but not control material, indicating the formation of Tau fibrils from disease brain extract (FIGS. 6A, 6B, 7A, 7B, and 8, scatter plots shown in the top row of each figure). The proteins were sedimented and analyzed by SDS-PAGE and Coomassie staining showing all protein in the pellet fraction indicating fibril formation as opposed to the supernatant fraction which would indicate protein in monomer form (FIGS. 6A, 6B, 7A, 7B, and 8, staining results shown in the bottom row of each figure). The results are shown through the 10⁻⁴, 10⁻⁶, 10⁻⁸, 10⁻¹⁰, and 10⁻¹² dilution generations.

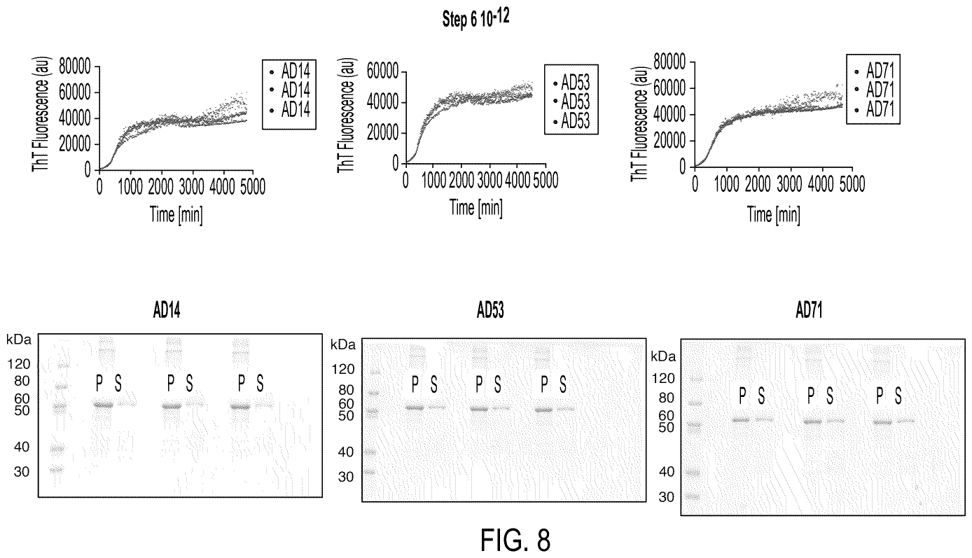

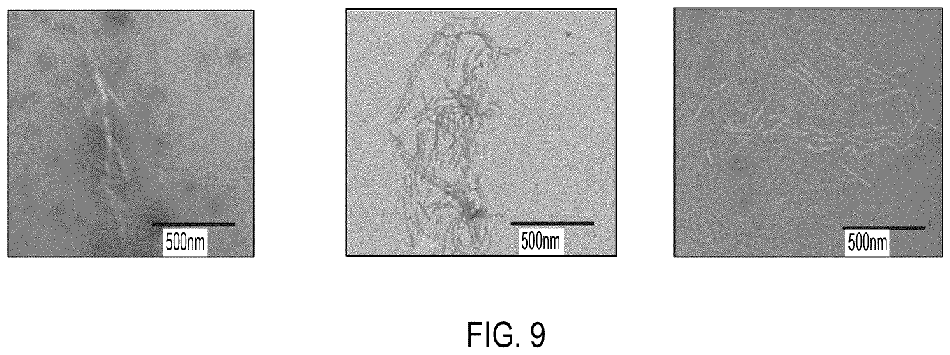

FIG. 9 shows TEM images illustrating the formation of disease fibrils from brain tissue extract as corresponding to the results shown in FIG. 8. In particular, the TEM images show disease fibrils formed from 10⁻¹² dilution generation.

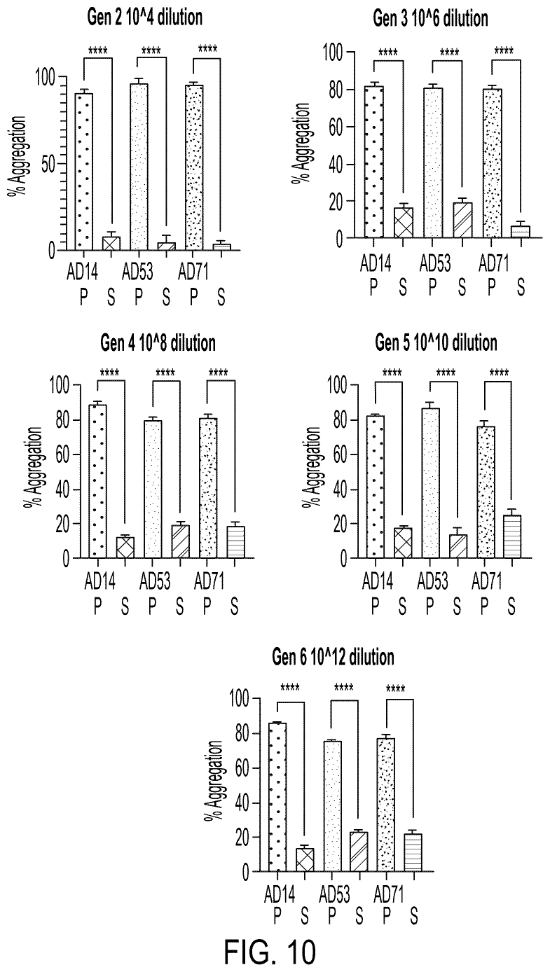

FIG. 10 shows bar graphs illustrating the quantified SDS-PAGE and Coomassie staining results of FIGS. 6A-8. More specifically, the bar graphs correspond to generations 2-6 of gel quantifications and statistical comparison using the one-way ANOVA test, with **** P≤0.0001. Error bars represent means±SD.

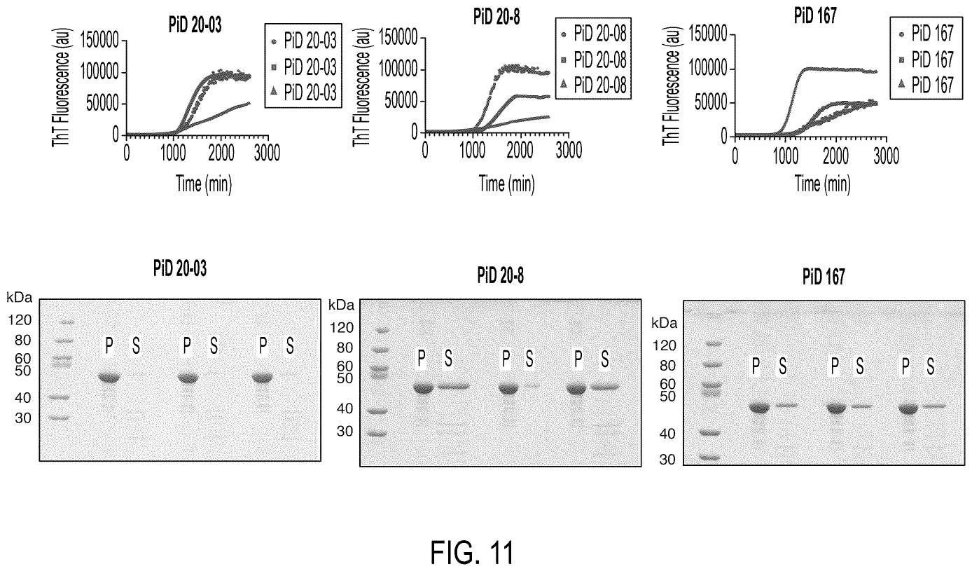

FIG. 11 shows experimental results from Step 1 of the multistep Tau amplification assay of FIGS. 1 and 2, shown here to illustrate the formation of Tau fibrils from brain extract of a Pick's Disease (PiD) patient, in accordance with various embodiments disclosed herein. Particularly, full length 3R Tau mutant monomer 23 wt is added to PiD disease brain extract under RT-QuIC assay conditions at 37° C. In the illustrated example, the binding of Thioflavin T to Tau fibrils results in fluorescence in disease material but not control material, indicating the formation of Tau fibrils from disease brain extract (FIG. 11, top row). The proteins were sedimented and analyzed by SDS-PAGE and Coomassie staining. The disease brain showed most of the protein in the pellet fraction as opposed to the supernatant fraction, indicating that the protein was mostly in its fibrillar form (FIG. 11, bottom row).

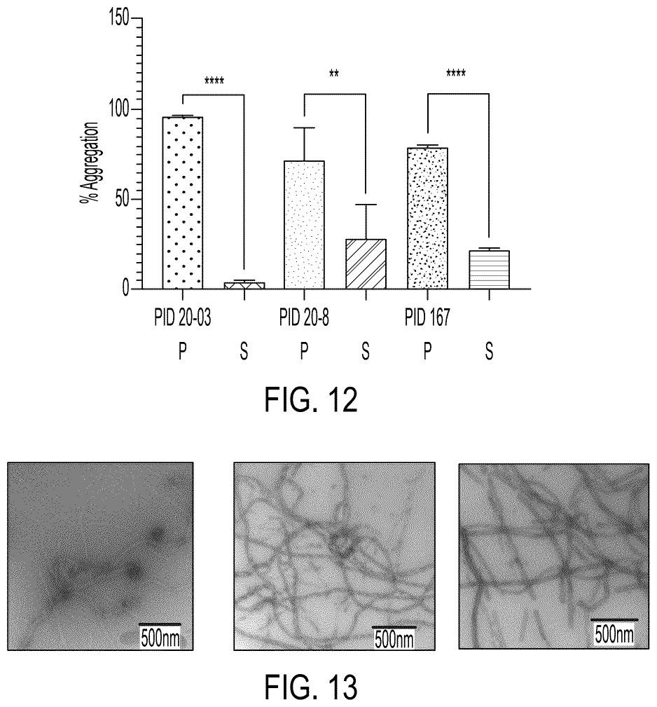

FIG. 12 shows a bar graph illustrating the quantified SDS-PAGE and Coomassie staining results of FIG. 11. In the present case, bar graph representations of the results of a quantification of the PiD gels and statistical comparison using one-way ANOVA test, with ****P≤0.0001 for two brains and one with **P≤0.001, is shown in accordance with various embodiments disclosed herein. Error bars again represent means±SD.

FIG. 13 shows TEM images illustrating the formation of disease fibrils from brain tissue extract as corresponding to the results shown in FIGS. 11 and 12. Particularly, TEM images show disease fibrils formed from the brain material.

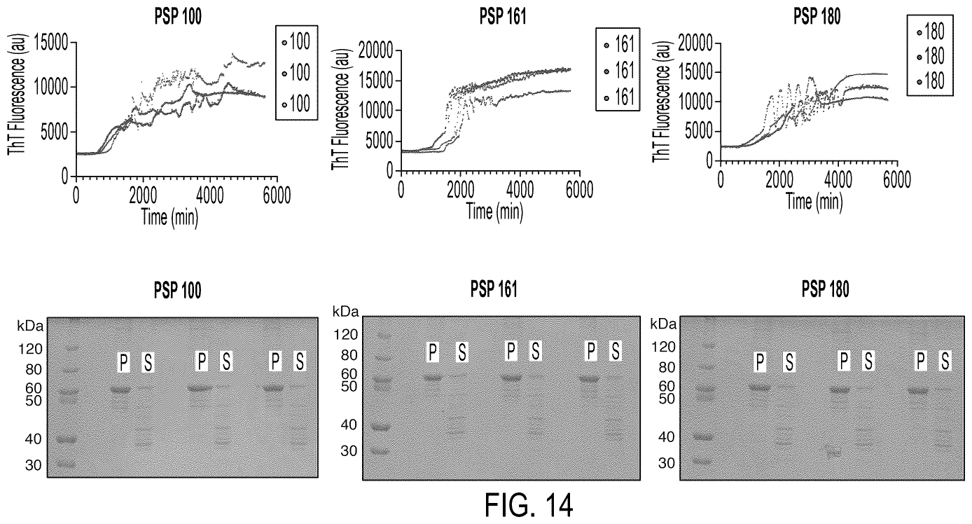

FIG. 14 shows experimental results from Step 1 of the multistep Tau amplification assay of FIGS. 1 and 2, shown here to illustrate the formation of Tau fibrils from brain extract of a Progressive Supranuclear Palsy (PSP) patient, in accordance with various embodiments disclosed herein. More specifically, full length 4R Tau mutant monomer P301S is added to PSP disease brain extract under RT-QuIC assay conditions at 37° C. The binding of Thioflavin T to Tau fibrils results in fluorescence in disease material indicating the formation of Tau fibrils from disease brain extract (FIG. 14, top row). The proteins were sedimented and analyzed by SDS-PAGE and Coomassie staining. The disease extract showed most of the protein in the pellet fraction as opposed to the supernatant fraction indicating that the protein was mostly in its fibrillar form (FIG. 14, bottom row).

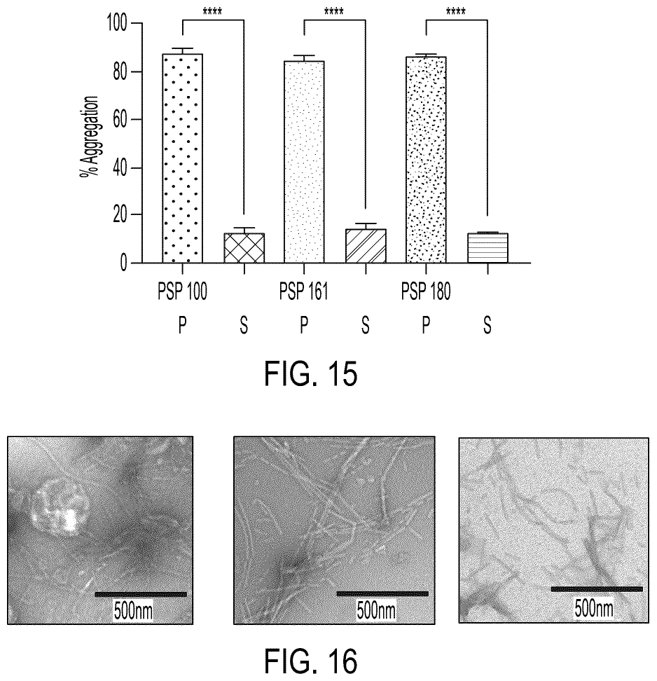

FIG. 15 shows a bar graph illustrating the quantified SDS-PAGE and Coomassie staining results of FIG. 14. Particularly, a quantification of the PSP gels and statistical comparison of the results were performed using one-way ANOVA test, with **** P≤0.0001 and error bars representing means±SD.

FIG. 16 shows TEM images illustrating the formation of disease fibrils from brain tissue extract as corresponding to the results shown in FIG. 14. As shown in FIG. 16, the TEM images show disease fibrils formed from the brain material.

Results similar to those illustrated above have been obtained from brain extract from an Alzheimer's Disease (AD) patient. For example, seeding of amplified tau fibrils in HEK293 cells has been demonstrated by adding full length 2N4R wild type 4R tau monomer to Alzheimer's disease brain extract under RT-QuIC assay conditions at 37° C. Amplified fibrils from the RT-QuIC reactions were then seeded into HEK293 cells and observed under confocal fluorescence microscopy to assess puncta formation, which is indicative of aggregation in cells. Control cells without any amplified fibrils showed no puncta fluorescence and a full plate of cells. Cells with amplified fibrils from AD brain showed puncta as well as a reduced number of cells indicating some toxicity from the seeds added.

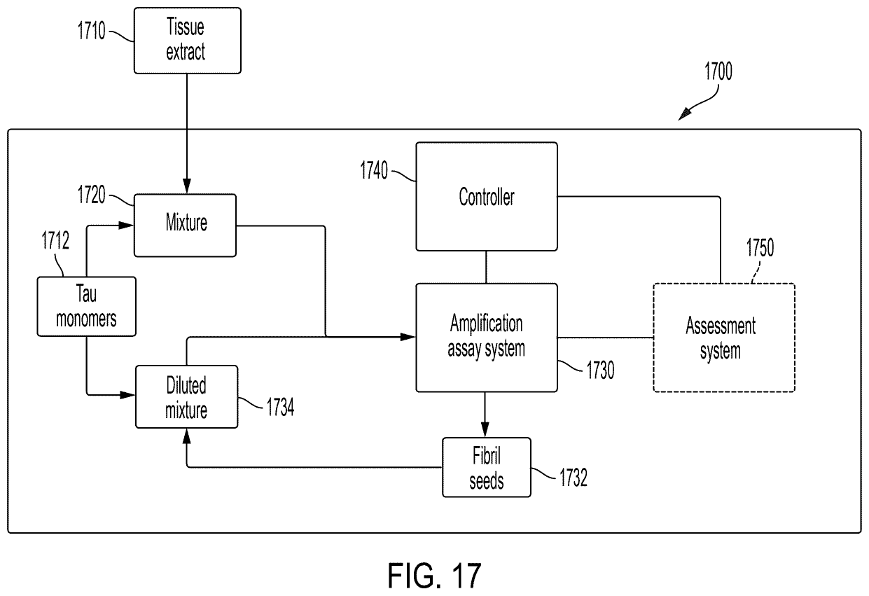

FIG. 17 illustrates an exemplary system configured to perform the multistep Tau amplification methods described herein. The system may include an amplification assay subsystem configured to perform incubation, agitation, and seed-generation steps; an assessment subsystem for monitoring assembly formation; and a controller configured to automate and coordinate amplification cycles. In some embodiments, the system may be implemented in microplate, tube-based, microfluidic, continuous-flow, or automated high-throughput formats.

FIG. 17 shows a system suitable for performing the multistep Tau amplification assay, in accordance with embodiments. As shown in FIG. 17, a multistep Tau amplification system 1700 is configured to receive tissue extracts 1710, such as described above, which becomes the initial fibril seeds for the amplification process. Tissue extract 1710 is mixed with Tau monomers 1712, which serve as the substrate for the subsequent amplification process. Mixing of the tissue extracts with the Tau monomer results in a mixture 1720, which is then fed into an amplification assay system 1730. Amplification assay system 1730 includes components, such as a quaking system, an incubator, a sonication system, and others (not explicitly shown in FIG. 17), which performs portions of the amplification assay process such as illustrated in FIG. 2 above. Amplification assay system 1730 may also be configured to accommodate and process mixture 1720 in a standard laboratory format, such as a 96-well plate or another sample platform. In an example, amplification assay system 1730 may include a RT-QuIC assay system as discussed above.

In embodiments, amplification assay system 1730 performs relevant steps of process of 200 illustrated in FIG. 2 to generate a solution including new fibril seeds 1732. New fibril seeds 1732 are again mixed with Tau monomers 1712 to form a diluted mixture 1734. Diluted mixture 1734 may again be fed into amplification assay system 1730 for another cycle of the amplification process.

Continuing to refer to FIG. 17, in embodiments, multistep Tau amplification system 1700 may further include a controller 1740 including one or more processors and memory on which instructions may be stored for controlling the operations of amplification assay system 1710. Multistep Tau amplification system 1700 also optionally includes an assessment system 1750 for performing real-time or post amplification assessment of the output from amplification assay system 1710. Furthermore, controller 1740 may be configured to control the operation of assessment system 1750.

As an example, assessment system 1750 may be used to monitor the Tau fibril growth and/or breakage in amplification assay system 1730. Additionally, as discussed above with respect to FIG. 2, controller 1740 may control assessment system 1750 to perform the determination whether the output from amplification assay system 1730 includes the sufficient dilution of the original tissue extract based on the results of assessment performed by assessment system 1730 to repeat certain steps in amplification process 200 of FIG. 2 as needed. The memory included in controller 1740 may further be configured to store data, such as various parameters of the amplification assay and the sample contents.

In some embodiments, the components of system 1700 are implemented as integrated or distributed modules, and one or more components may be combined into a single device or implemented using software-controlled instrumentation. The system may be configured to execute one or more steps of the serial amplification methods described herein, including mixing, incubation, agitation, seed generation, assessment, and iterative cycling, in a partially or fully automated manner.

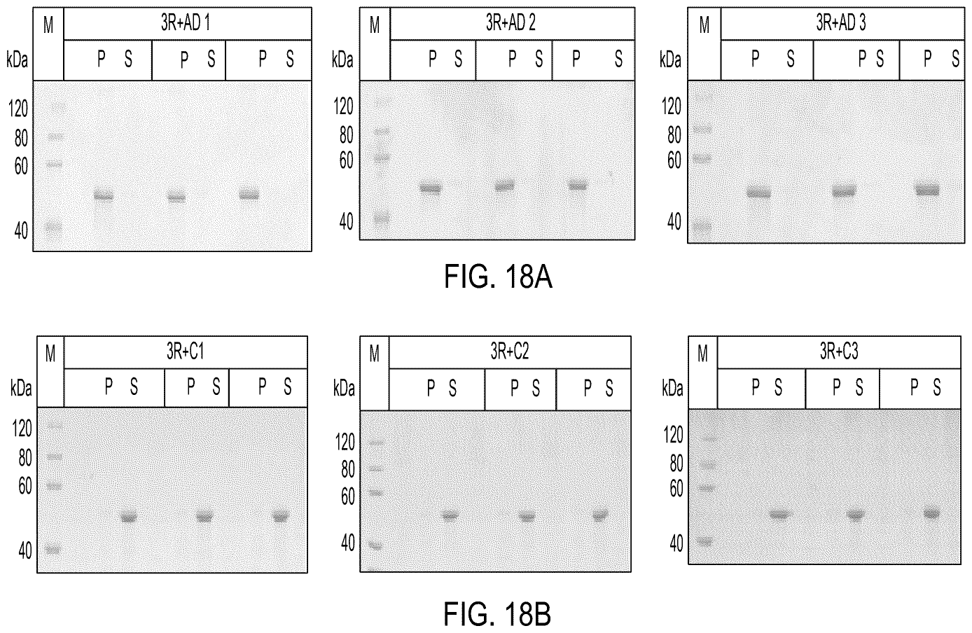

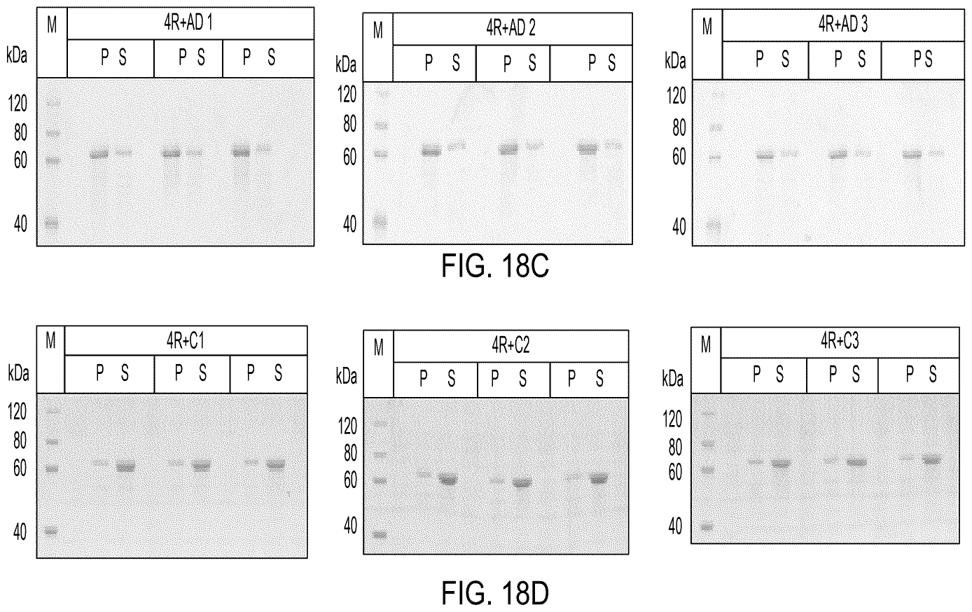

Additional experimental and characterization results are shown in FIGS. 18A-24C-2, which illustrate further embodiments and performance characteristics of the co-factor-free serial amplification methods described herein. FIGS. 18A-18D illustrate representative sedimentation analyses demonstrating aggregation of full-length Tau monomers in the presence of Alzheimer's disease brain homogenates (AD1-AD3), but not in the presence of control brain homogenates, confirming reproducible and seed-dependent Tau assembly under co-factor-free amplification conditions.

FIGS. 18A-18D show experimental results demonstrating serial amplification of Tau assemblies seeded from Alzheimer's disease (AD) biological material using recombinant 3R Tau monomeric substrate across multiple amplification generations, in accordance with various embodiments disclosed herein. In the illustrated embodiments, AD-derived Tau seed material is contacted with monomeric Tau under conditions permissive for seed-dependent Tau assembly growth to produce amplified Tau assemblies. At least a portion of the amplified Tau assemblies is processed (e.g., by sonication or other mechanical disruption) to generate Tau seed material for further amplification. The amplification cycle is repeated one or more times to produce serially amplified Tau assemblies. In some embodiments, amplification is monitored by fluorescence kinetics (e.g., Thioflavin T fluorescence) and/or biochemical sedimentation to assess conversion of monomeric Tau into pelletable Tau assemblies.

In exemplary embodiments, Tau monomer conversion into aggregated Tau assemblies is confirmed using biochemical sedimentation followed by gel electrophoresis. For example, after mixing recombinant full-length 3R Tau monomers or full-length 4R Tau monomers with Alzheimer's disease (AD) brain homogenates from multiple independent subjects (AD1-AD3) or with control brain homogenates (C1-C3), and subjecting the mixtures to seed-dependent amplification conditions (e.g., RT-QuIC conditions), the reaction products are sedimented to separate pellet (P) and supernatant (S) fractions. The fractions are analyzed by SDS-PAGE and Coomassie staining to evaluate distribution of Tau between aggregated and soluble states. In embodiments, 3R Tau migrates at an apparent molecular weight of approximately 52 kDa and 4R Tau migrates at an apparent molecular weight of approximately 64 kDa, with a protein marker (M) used for reference. In some embodiments, the experiments are performed with technical replicates (e.g., triplicates). The sedimentation results may be quantified (e.g., densitometry) to determine the relative distribution of Tau in pellet versus supernatant fractions and to support quantitative comparisons of aggregation efficiency.

In some embodiments, FIGS. 18A-18D further demonstrates reproducibility of conversion across independent biological samples and/or independent amplification reactions. For example, independent AD-derived seed sources may yield consistent conversion of monomeric Tau to amplified Tau assemblies under co-factor-free conditions, thereby supporting robustness of the amplification methods disclosed herein.

FIGS. 19A-1-19C show experimental results demonstrating serial amplification of Tau assemblies seeded from AD biological material using recombinant Tau monomeric substrates across multiple amplification generations, in accordance with various embodiments disclosed herein. In some embodiments, AD-derived Tau assemblies support amplification of both 3R and 4R Tau isoforms, consistent with mixed-isoform Tauopathies. In some embodiments, the results of FIGS. 19A-1-19C show that amplification proceeds efficiently across multiple generations, including at advanced dilution generations in which non-Tau components from the initial biological material are substantially diluted, thereby supporting co-factor-free propagation driven by Tau assemblies themselves.

In various exemplary embodiments as shown in FIGS. 19A-1-19A-2, serial amplification of Tau assemblies is demonstrated over multiple amplification cycles without requiring aggregation-promoting co-factors. For example, Tau assemblies amplified from Alzheimer's disease (AD)-derived seed material may be serially propagated through multiple generations (e.g., Generations 2-6) using recombinant full-length 3R Tau monomers as substrate under seed-dependent amplification conditions (e.g., RT-QuIC conditions), e.g., as illustrated in FIGS. 26A-1, 26A-2, 26A-3, 26A-4, and 26A-5. Following one or more amplification cycles, reaction products from each generation may be subjected to sedimentation to separate pellet (P) and supernatant (S) fractions, and the fractions may be analyzed by SDS-PAGE and Coomassie staining to assess monomer-to-assembly conversion. In some embodiments, serial amplification is performed using independent biological seed sources (e.g., AD1-AD3), thereby demonstrating reproducibility across biological replicates. In the illustrated gels, gels within a row correspond to biological triplicates, and gels within a column correspond to successive amplification generations. In embodiments, a protein marker (M) may be included, and 3R Tau migrates at an apparent molecular weight of approximately 52 kDa. Band intensities may be quantified (e.g., densitometry) to determine the distribution of Tau protein between pellet and supernatant fractions across generations, thereby supporting assessment of conversion efficiency and serial propagation capability, e.g., as illustrated in FIGS. 26B-1, 26B-2, 26B-3, 26B-4, and 26B-5.

In exemplary embodiments, serial amplification of Tau assemblies is demonstrated over multiple amplification cycles without requiring aggregation-promoting co-factors using recombinant 4R Tau monomers as substrate. For example, Tau assemblies amplified from Alzheimer's disease (AD)-derived seed material may be serially propagated through multiple generations (e.g., Generations 2-6) using recombinant full-length 4R Tau monomers under seed-dependent amplification conditions (e.g., RT-QuIC conditions), as illustrated in FIGS. 26C-1, 26C-2, 26C-3, 26C-4, and 26C-5C. Following one or more amplification cycles, reaction products from each generation may be subjected to sedimentation to separate pellet (P) and supernatant (S) fractions, and the fractions may be analyzed by SDS-PAGE and Coomassie staining to assess monomer-to-assembly conversion, as shown in FIGS. 19B-1-19B-2. In the illustrated gels, gels within a row correspond to biological triplicates (e.g., AD1-AD3), and gels within a column correspond to successive amplification generations (e.g., Generations 2-6). In embodiments, a protein marker (M) may be included, and 4R Tau migrates at an apparent molecular weight of approximately 64 kDa. Band intensities may be quantified (e.g., densitometry) to determine the distribution of Tau protein between pellet and supernatant fractions across generations, thereby supporting assessment of conversion efficiency and serial propagation capability, as illustrated in FIGS. 26D-1, 26D-2, 26D-3, 26D-4, and 26D-5.

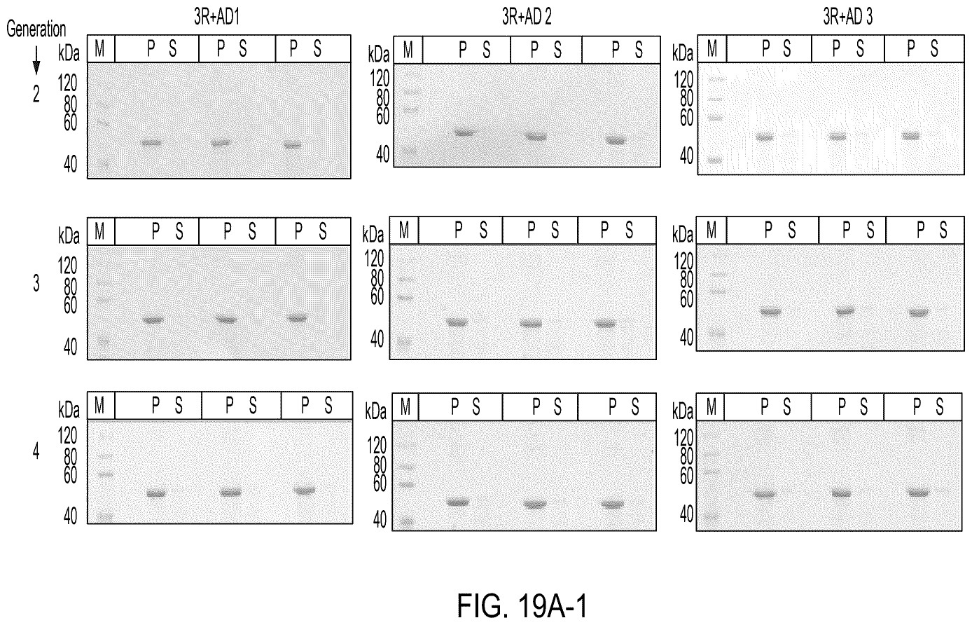

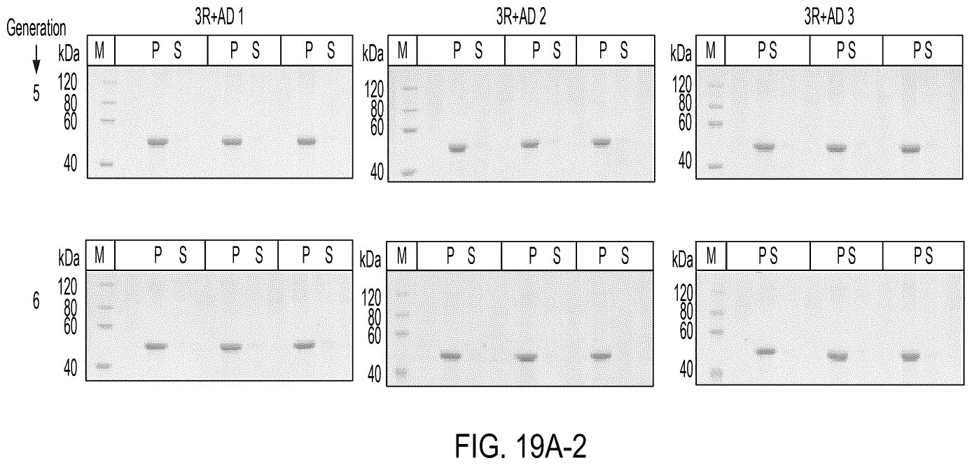

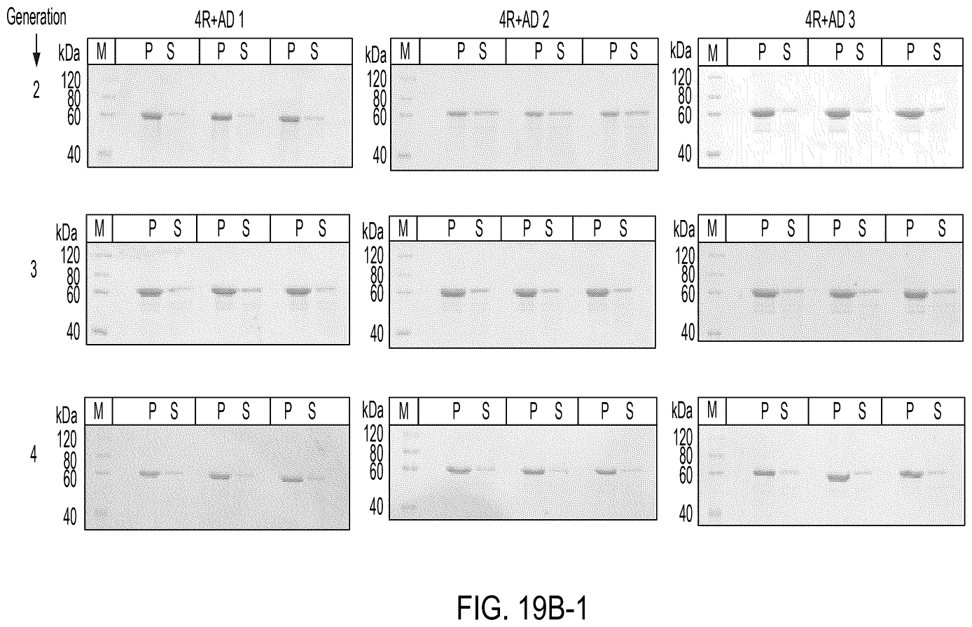

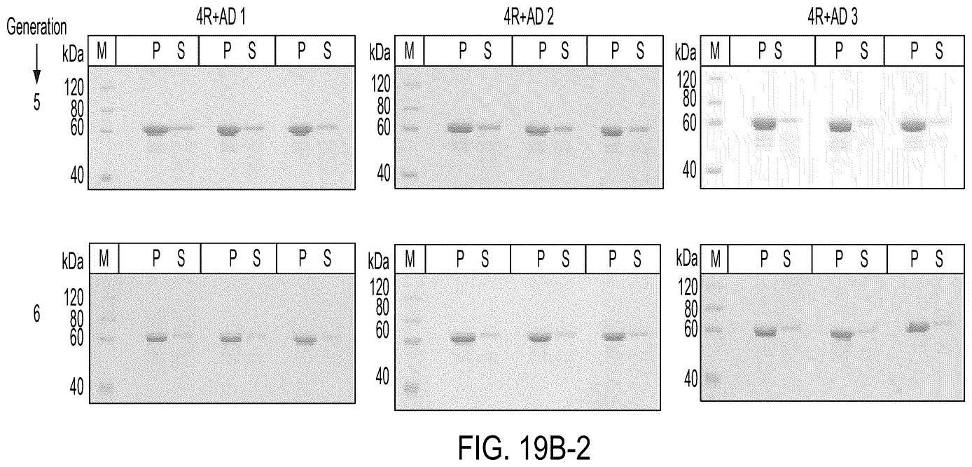

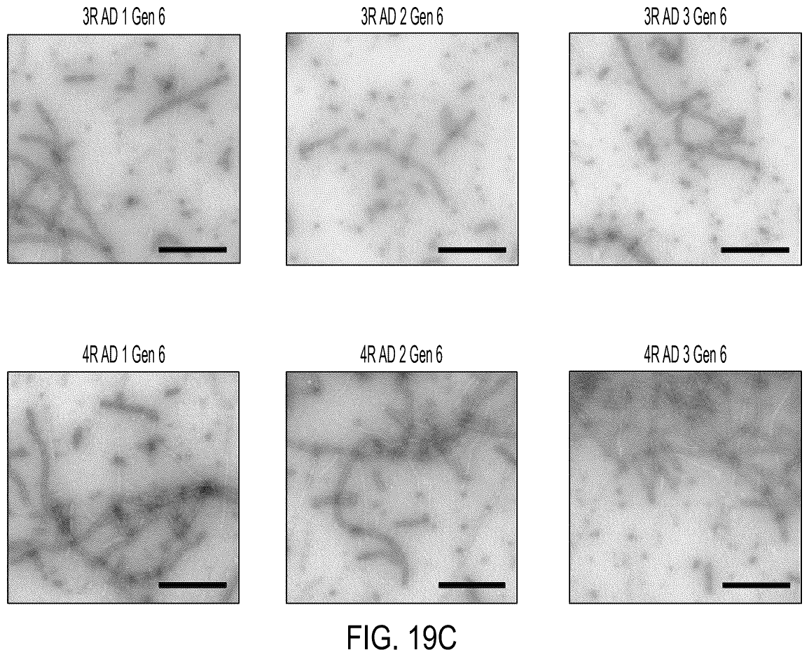

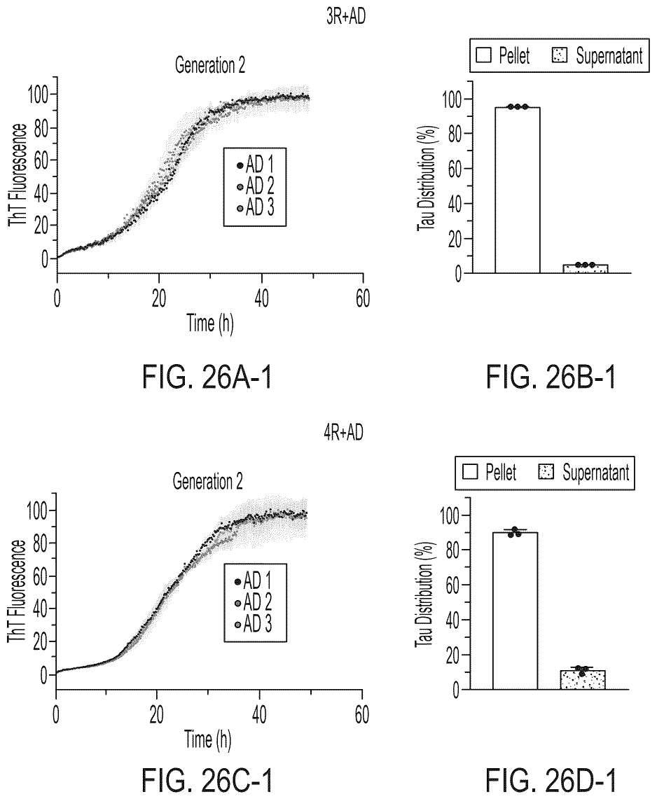

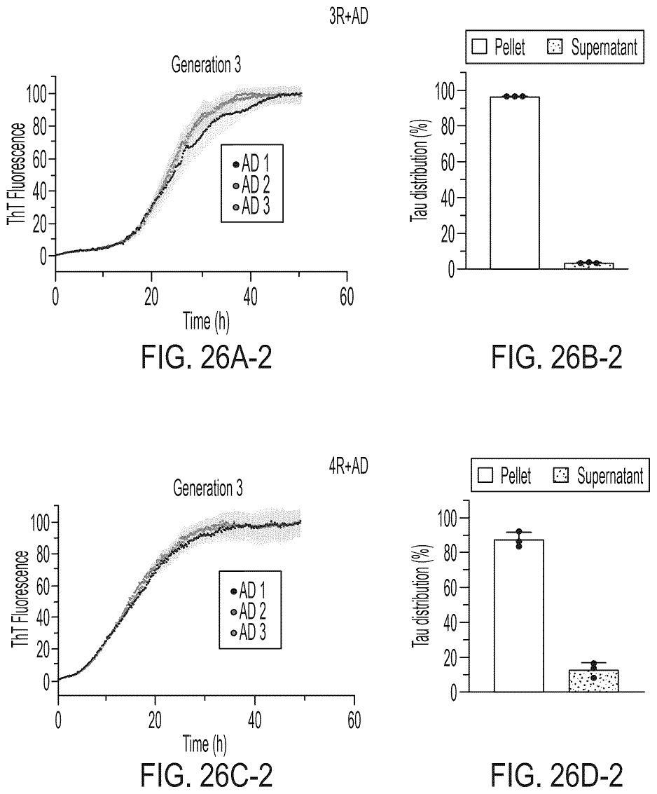

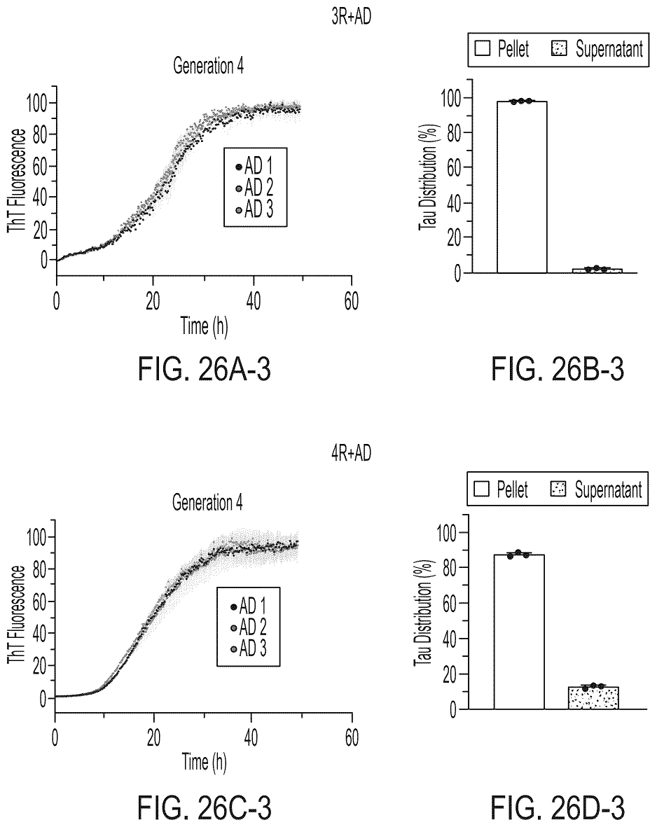

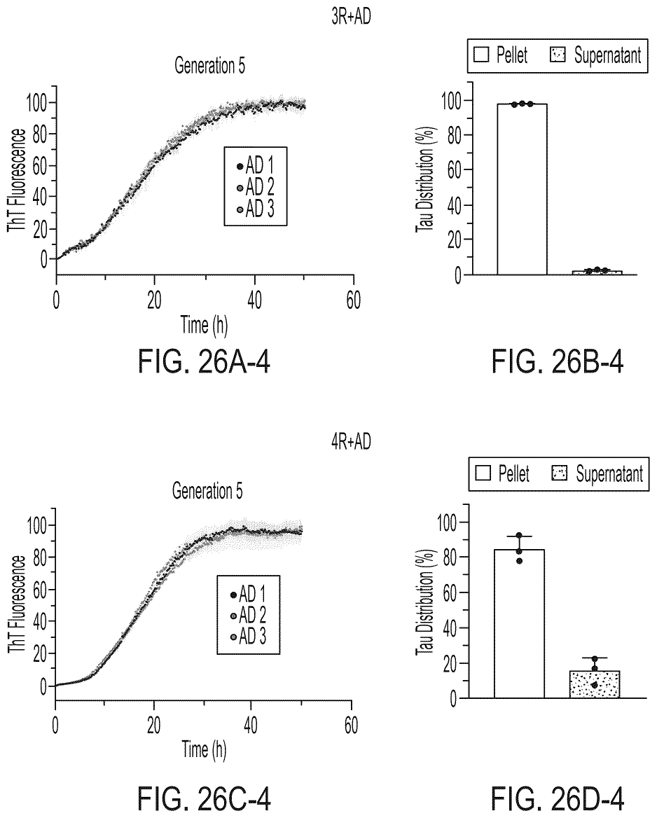

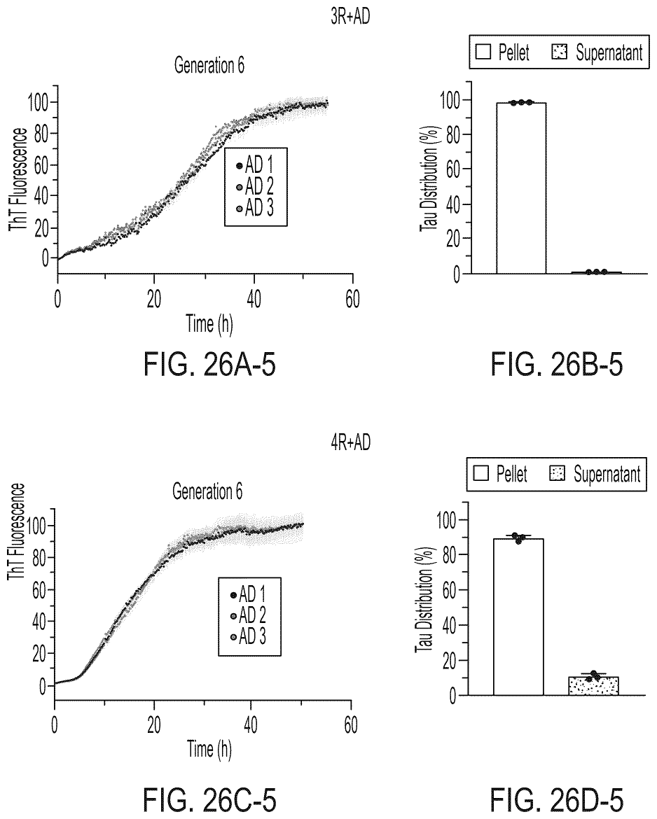

In exemplary embodiments, Tau assemblies generated by serial amplification are confirmed to be fibrillar by electron microscopy. For example, following a final amplification generation (e.g., Generation 6 as described with reference to FIGS. 26A-5, 26B-5, 26C-5, and 26D-5), serially amplified Tau assemblies produced using recombinant 3R Tau monomers and recombinant 4R Tau monomers are imaged by negative-stain transmission electron microscopy (TEM), thereby confirming fibrillar morphology. In some embodiments, the imaged 3R Tau fibrils and 4R Tau fibrils are descended from independent AD-derived seed sources (e.g., AD1-AD3). In embodiments, representative TEM images are shown in FIG. 19C, with scale bars of 500 nm.

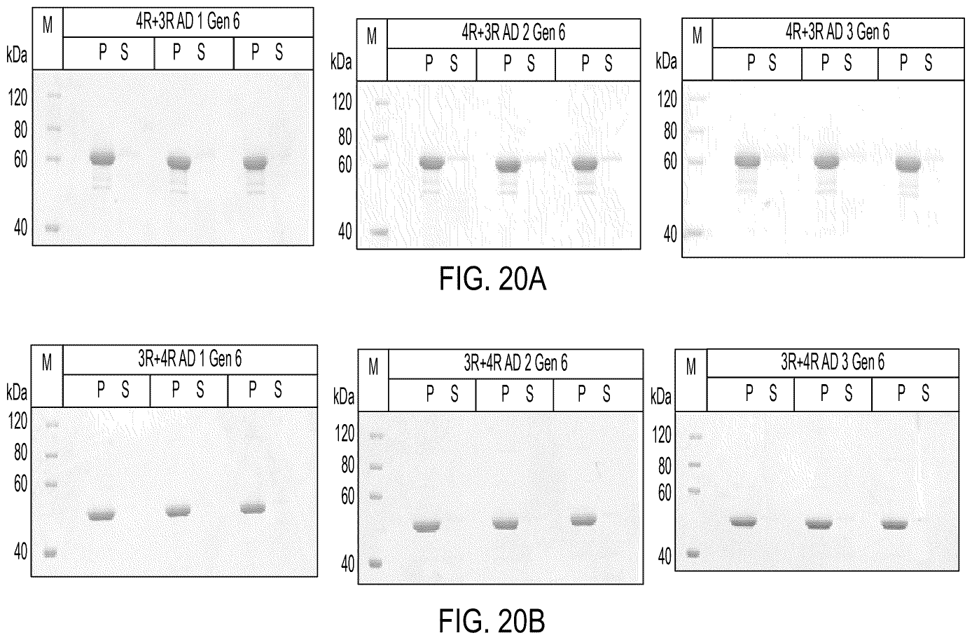

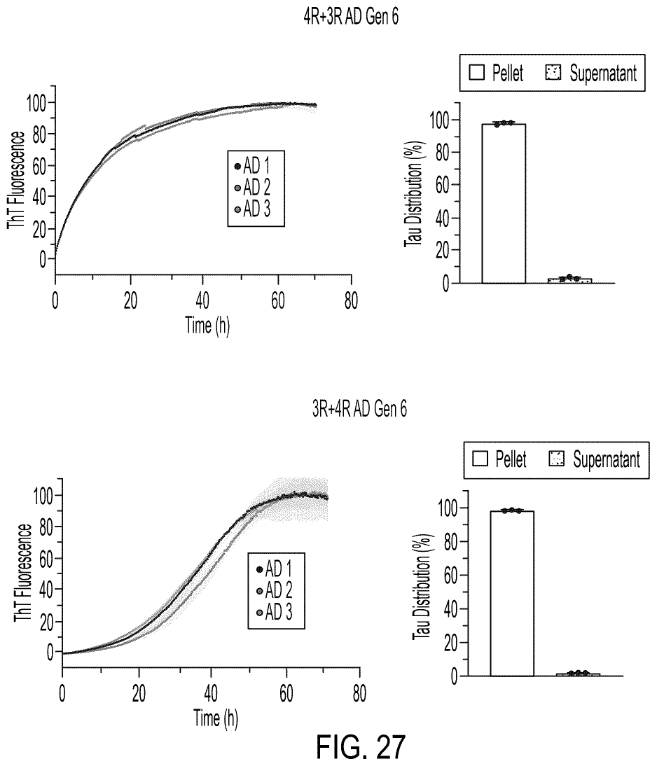

In exemplary embodiments, serially amplified Tau assemblies retain cross-seeding activity between distinct Tau isoforms. FIGS. 20A-20B illustrate SDS-PAGE and Coomassie staining analysis of cross-seeded Tau assemblies produced from Generation 6 fibrils using a seed-dependent amplification protocol (e.g., RT-QuIC conditions), as described with reference to FIG. 27 (scatter plots). In embodiments, recombinant 4R Tau monomers are cross-seeded with serially amplified 3R Tau fibrils and, conversely, recombinant 3R Tau monomers are cross-seeded with serially amplified 4R Tau fibrils. Reaction products are sedimented to separate pellet (P) and supernatant (S) fractions and analyzed by SDS-PAGE and Coomassie staining to assess conversion of monomeric Tau into aggregated Tau assemblies. In embodiments, 3R Tau migrates at an apparent molecular weight of approximately 52 kDa and 4R Tau migrates at an apparent molecular weight of approximately 64 kDa, with a protein marker (M) included for reference. In some embodiments, cross-seeding is demonstrated using independent AD-derived seed lineages (e.g., AD1, AD2, and AD3). Band intensities may be quantified (e.g., densitometry) to determine distribution of Tau between pellet and supernatant fractions, as illustrated in FIG. 27 (bar graphs), thereby supporting that serially amplified Tau assemblies retain cross-seeding capabilities.

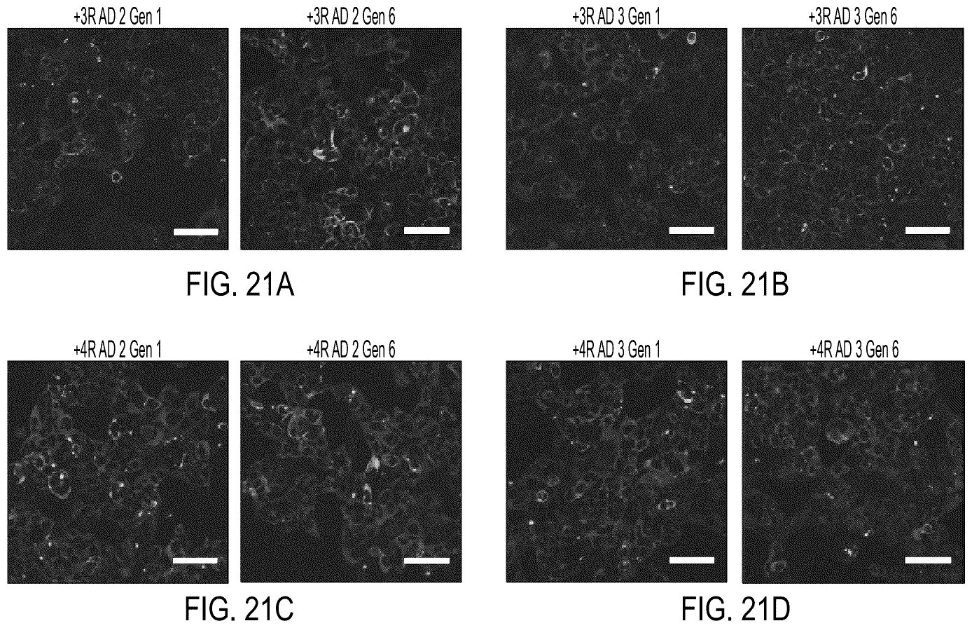

In exemplary embodiments, serially amplified Tau assemblies exhibit cellular seeding activity and induce intracellular Tau aggregation. FIGS. 21A-21D illustrate representative fluorescence microscopy images of monoclonal HEK293 cells expressing htau40P301S (a 2N4R Tau variant) fused to enhanced yellow fluorescent protein (EYFP) at the C-terminus following introduction of Tau seed material derived from serial amplification reactions. In embodiments, Tau seeds corresponding to Generation 1 and Generation 6 amplification products are introduced into the cells and incubated for approximately 24 hours at about 37° C. In some embodiments, the Tau seeds are descended from independent AD brain homogenate sources (e.g., AD2 and AD3) and amplified using either recombinant 3R Tau monomers (panels (a)-(b)) or recombinant 4R Tau monomers (panels (c)-(d)). In embodiments, the left panels depict Generation 1 seeds and the right panels depict Generation 6 seeds. In embodiments, intracellular aggregation is observed as punctate fluorescence consistent with seeded aggregation of Tau within cells, thereby demonstrating that Tau assemblies generated and propagated by the disclosed co-factor-free serial amplification methods retain functional seeding competence in a cellular assay. Scale bars are 40 micrometer (m).

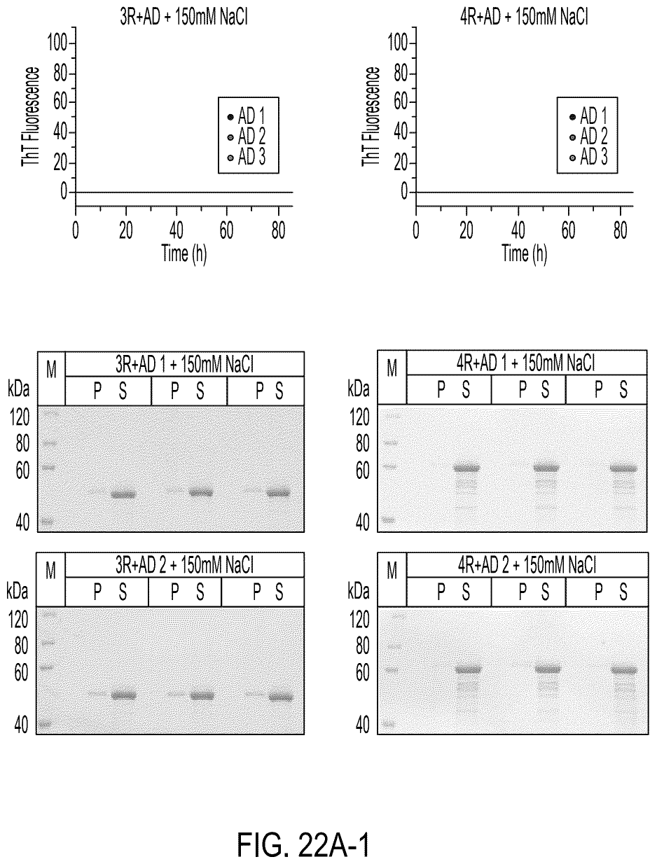

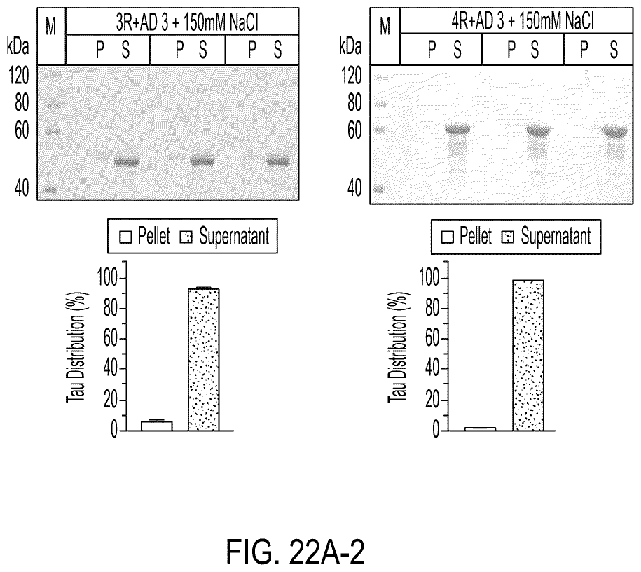

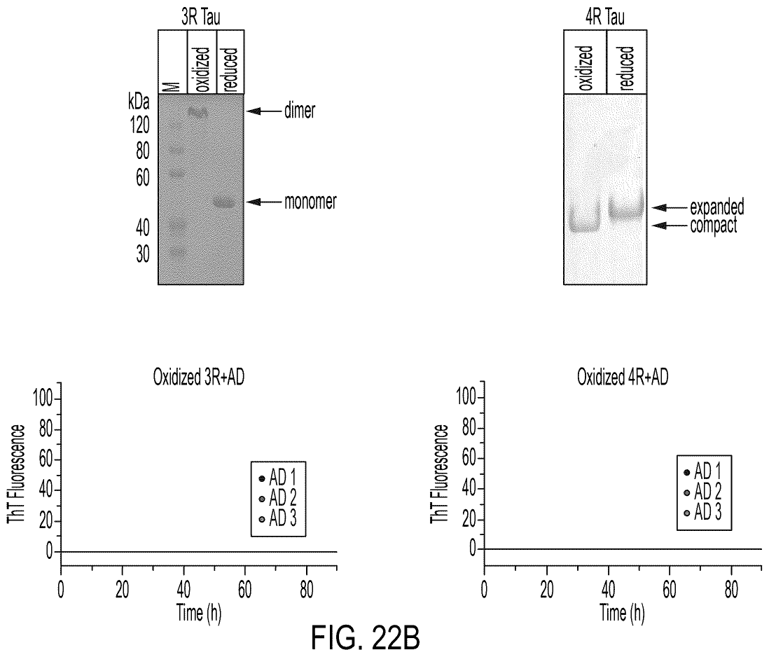

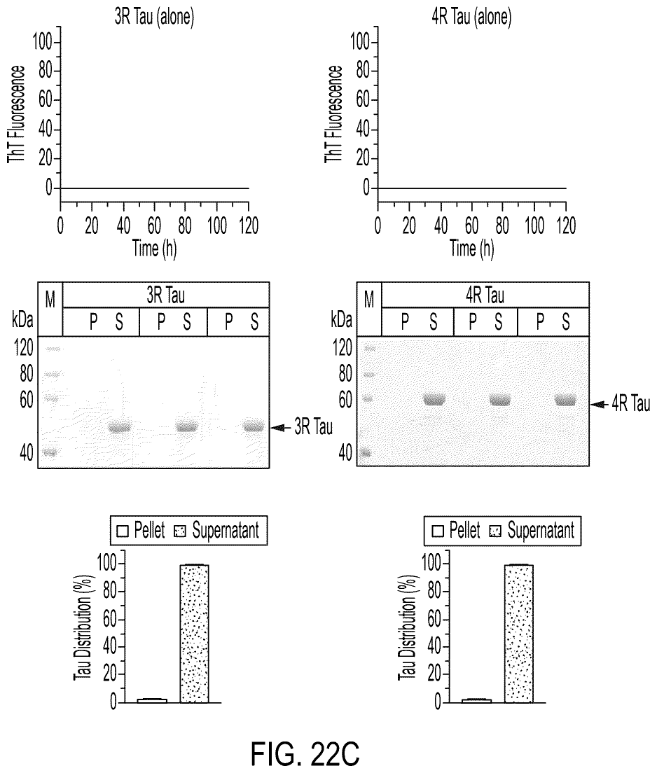

FIGS. 22A-1-22C show experimental results demonstrating that co-factor-free, seed-dependent Tau assembly growth is sensitive to reaction conditions and is dependent on the presence of seed material, in accordance with various embodiments disclosed herein. In some embodiments, increasing ionic strength inhibits seeded amplification (FIGS. 22A-1 and 22A-2), oxidation of Tau monomers inhibits recruitment onto pathological seeds (FIG. 22B), and monomeric Tau does not appreciably aggregate under the assay conditions in the absence of brain-derived seed material (FIG. 22C).

In some embodiments, ionic strength modulates seed-dependent Tau assembly growth. FIGS. 22A-1 and 22A-2 illustrate that increased salt concentration inhibits co-factor-free amplification of Tau assemblies seeded from Alzheimer's disease (AD) brain homogenate. Panels (a) and (b) show ThT fluorescence traces for reactions with 3R Tau and 4R Tau, respectively; panels (c) and (d) show corresponding SDS-PAGE/Coomassie gels; and panels (e) and (f) show quantified pellet-versus-supernatant distributions for 3R Tau and 4R Tau reactions, respectively. In embodiments, recombinant monomeric Tau protein (e.g., about 10 micromolar (μM) 3R Tau and/or 4R Tau) is combined with AD brain homogenate (e.g., AD1-AD3 at about 30 micrograms per milliliter (g/mL)) in sodium phosphate buffer (pH~7.4) containing about 150 millimolar (mM) NaCl and incubated at about 37° C. under RT-QuIC conditions in the presence of Thioflavin T (ThT). ThT fluorescence kinetics show little to no signal increase under elevated salt conditions for both 3R Tau and 4R Tau reactions, consistent with inhibited seeded assembly growth. Reaction products may be sedimented and analyzed by SDS-PAGE and Coomassie staining to assess distribution of Tau between pellet (P) and supernatant (S) fractions, and band intensities may be quantified and graphed to confirm limited conversion into aggregated assemblies under high-salt conditions. In some embodiments, experiments are performed in triplicate and results are reported as mean±standard deviation.

In some embodiments, the oxidation state of Tau modulates its ability to undergo seed-dependent assembly growth. FIG. 22B illustrates that AD brain homogenates fail to convert oxidized 3R and 4R Tau into aggregated Tau assemblies. In embodiments, Tau is oxidized (e.g., with about 1 mM hydrogen peroxide) to generate (i) intermolecular disulfide linkages between cysteine residues at position 291 of two 3R Tau monomers (forming 3R Tau dimers), and (ii) intramolecular disulfide linkages between cysteine residues at positions 291 and 322 of a 4R Tau monomer (forming compact 4R Tau monomers). The oxidized proteins may be purified by size exclusion chromatography, and oxidation may be verified by non-reducing SDS-PAGE for oxidized 3R Tau to identify dimers and by native-PAGE for oxidized 4R Tau to detect compact monomers, with reduced Tau (free thiol forms) included as controls. Oxidized Tau (e.g., about 10 μM) is then combined with AD brain homogenate (e.g., AD1-AD3 at about 30 g/mL) in sodium phosphate buffer (pH~7.4) without reducing agent and incubated at about 37° C. under RT-QuIC conditions in the presence of Thioflavin T (ThT). ThT fluorescence traces for oxidized 3R Tau and oxidized 4R Tau show little to no signal increase, consistent with inhibited seeded amplification. In some embodiments, experiments are performed in triplicate and results are reported as mean±standard deviation.

In some embodiments, Tau assembly growth is seed-dependent and does not occur spontaneously under amplification conditions in the absence of biological seed material. FIG. 22C illustrates that full-length 3R and 4R Tau monomers do not aggregate in the absence of brain homogenate. In embodiments, recombinant Tau monomers (e.g., about 10 μM) are incubated for approximately 120 hours at about 37° C. using RT-QuIC conditions in the presence of Thioflavin T (ThT). Panels (a) and (b) show ThT fluorescence traces for 3R Tau and 4R Tau, respectively, indicating little to no aggregation. Following incubation, samples are sedimented and analyzed by SDS-PAGE and Coomassie staining; panels (c) and (d) show representative gels for 3R Tau and 4R Tau, respectively, with M denoting a protein marker and P and S denoting pellet and supernatant fractions. Band intensities for pellet and supernatant fractions are quantified and graphed; panels (e) and (f) show quantification graphs for 3R Tau and 4R Tau, respectively. In some embodiments, experiments are performed in triplicate and results are reported as mean±standard deviation.

In some embodiments, as illustrated in FIGS. 22A-1-22C, the results show that increasing ionic strength (e.g., by addition of salt to a physiological or elevated concentration) reduces or prevents amplification, and/or that oxidation state of Tau monomers may affect incorporation into Tau assemblies. Such findings support embodiments in which amplification conditions are selected to promote accessibility of aggregation-prone motifs within Tau and to facilitate templated growth in the absence of added negatively charged co-factors.

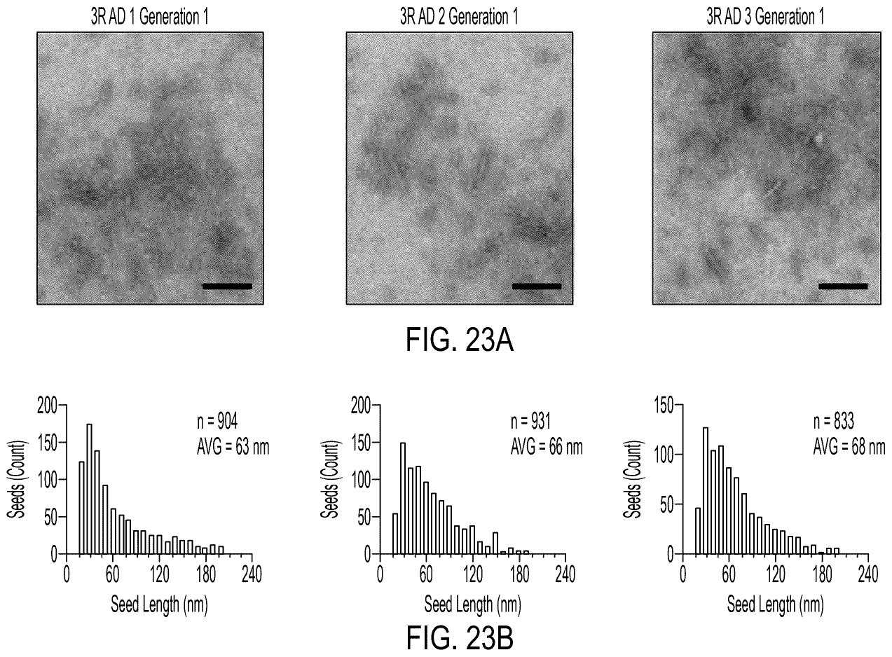

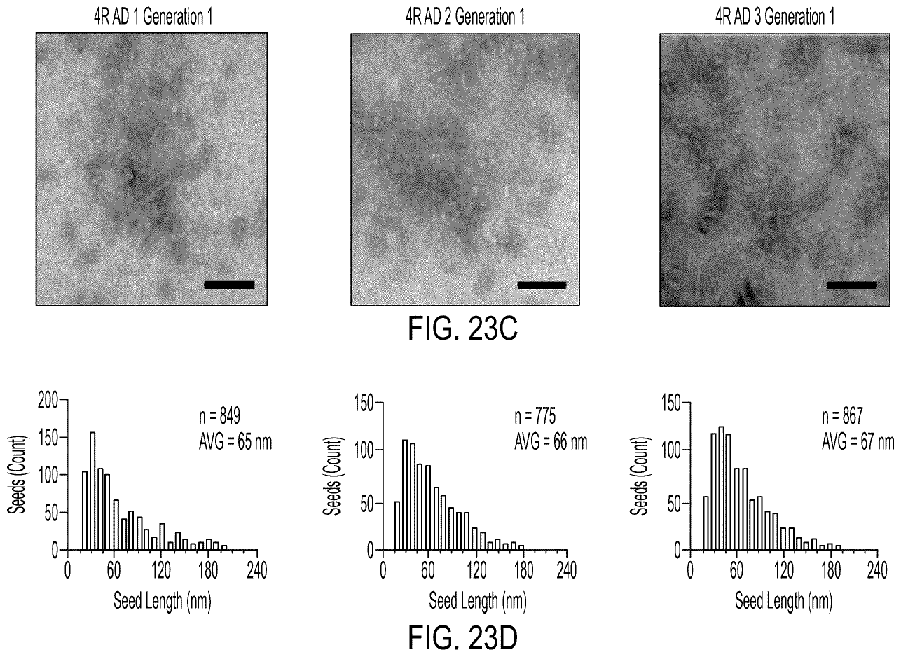

In exemplary embodiments, Tau fibril seeds generated for serial amplification exhibit reproducible size characteristics across independent biological seed sources. FIGS. 23A-23D show experimental results demonstrating that AD Generation 1 fibril seeds used for serial amplification have similar lengths. In embodiments, AD1-AD3 Tau fibrils generated by a first round of amplification (Generation 1) using either recombinant 3R Tau monomers or recombinant 4R Tau monomers as substrate (e.g., as described with reference to FIG. 1) are processed to generate seed material by tip-sonication for approximately 30 seconds on ice, and are then imaged by negative-stain transmission electron microscopy (TEM). In embodiments, representative TEM images are shown for seeds composed of 3R Tau (panel (a)) with corresponding length quantification (panel (b)), and representative TEM images are shown for seeds composed of 4R Tau (panel (c)) with corresponding length quantification (panel (d)). In embodiments, the left, center, and right panels correspond to seed populations descended from AD1, AD2, and AD3 homogenates, respectively. In embodiments, scale bars are 100 nm. In some embodiments, the similarity in seed lengths across AD1-AD3 supports reproducible seed generation suitable for iterative amplification across generations.

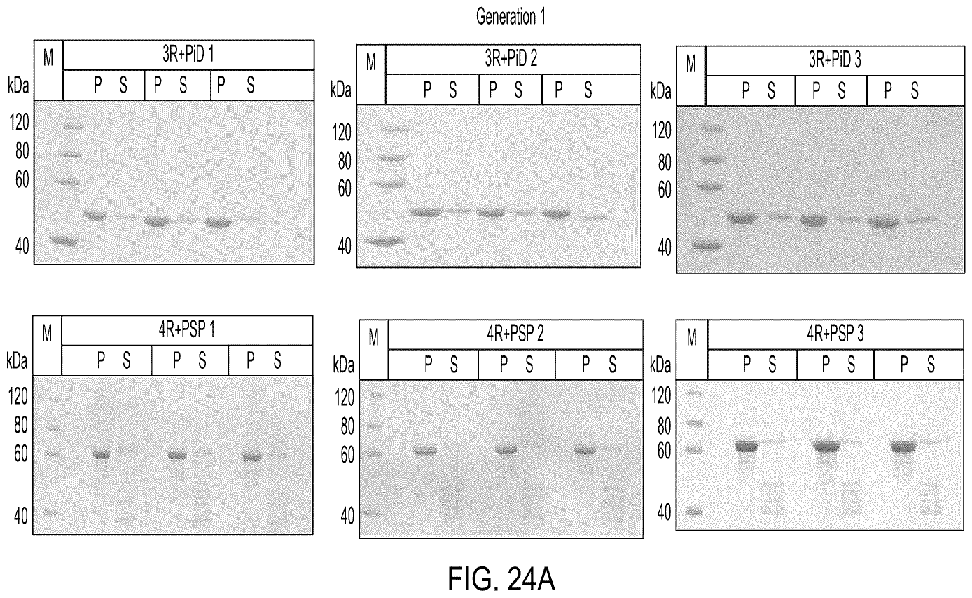

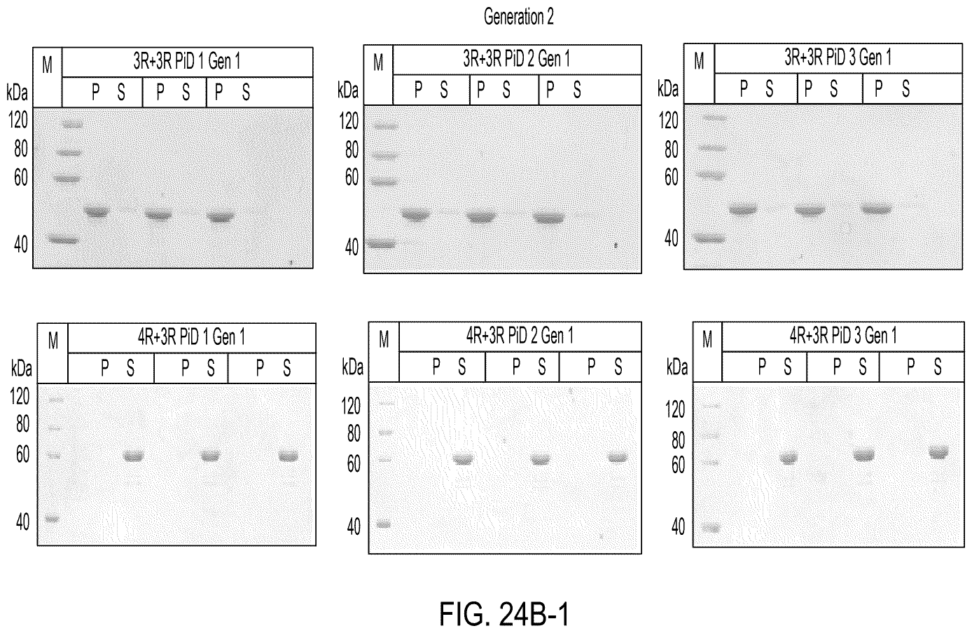

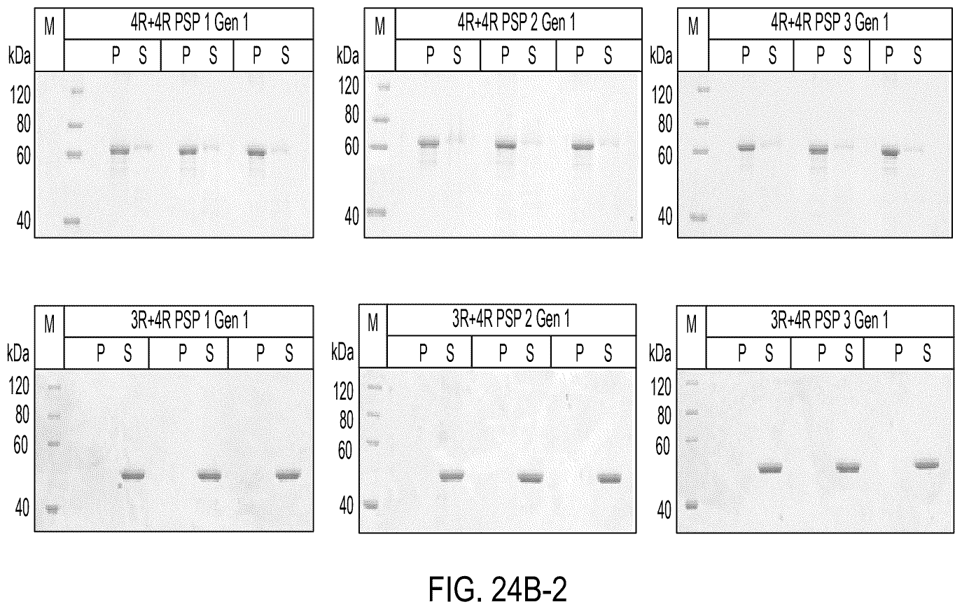

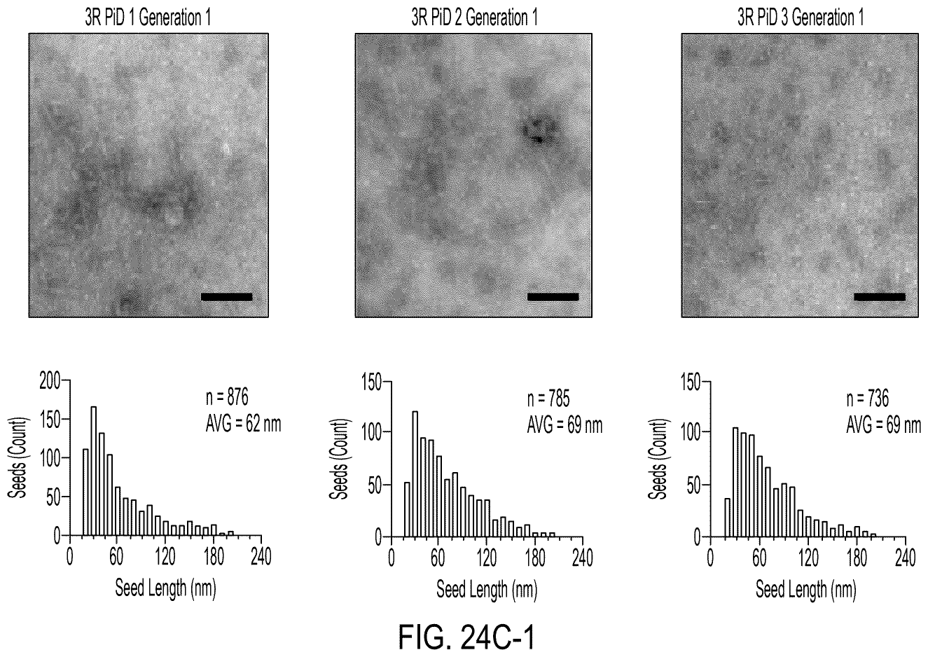

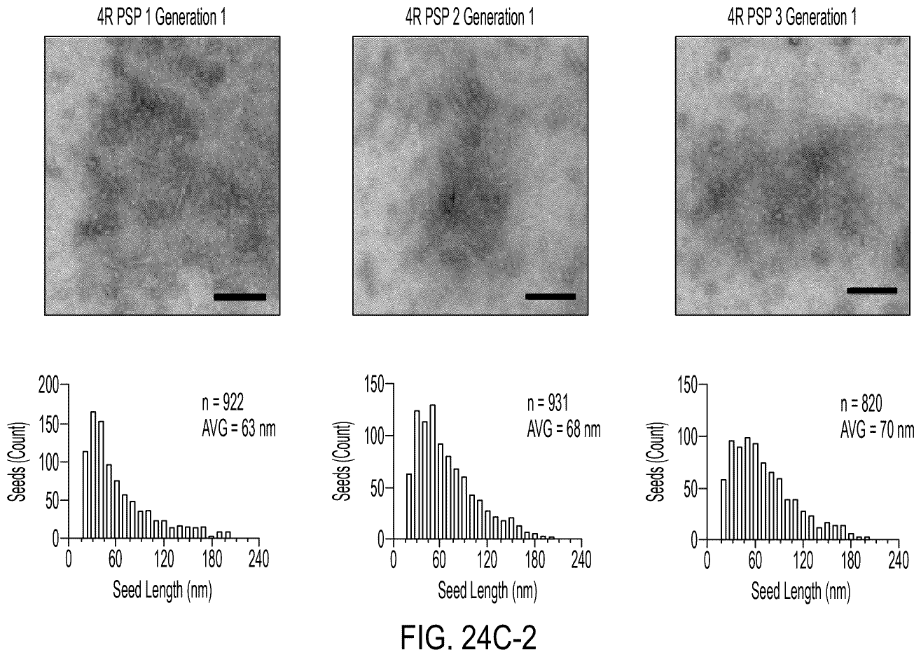

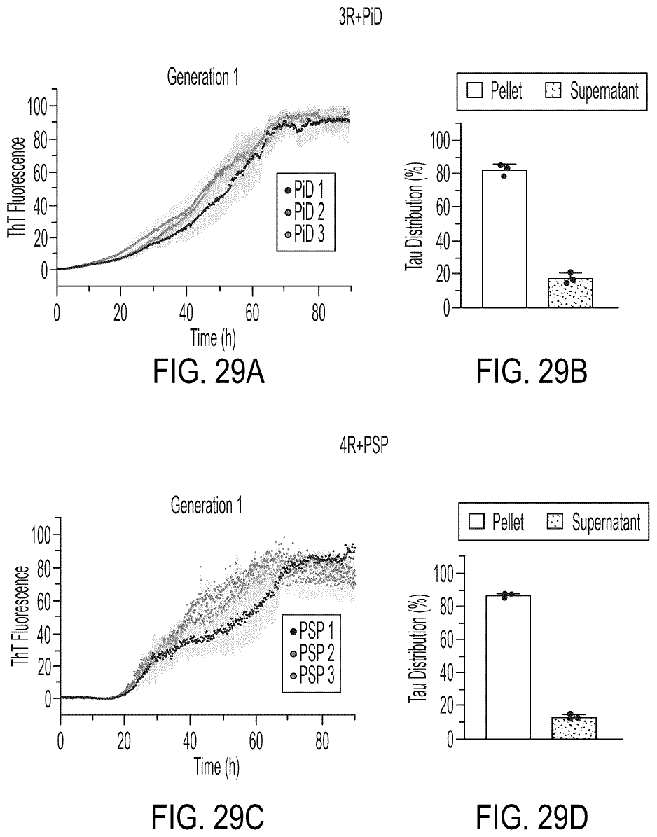

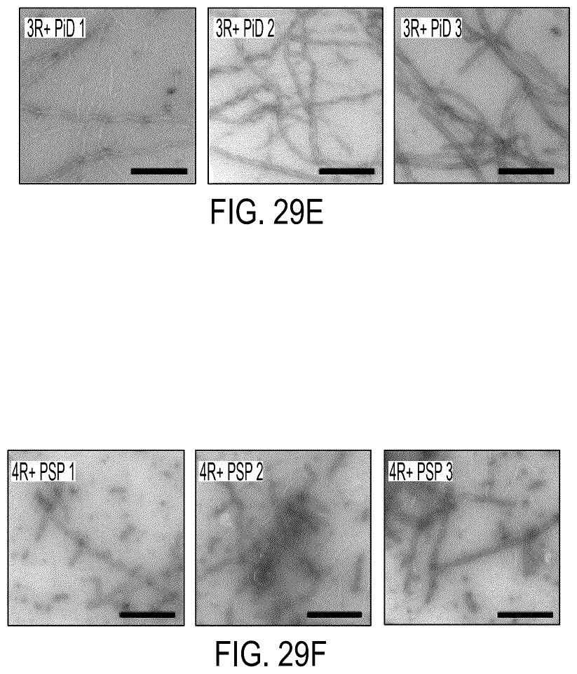

FIGS. 24A-24C-2 show experimental results demonstrating that the disclosed co-factor-free serial amplification methods are applicable to multiple tauopathy-derived biological sources, including Pick's disease (PiD) and progressive supranuclear palsy (PSP), and that the resulting Tau assemblies retain disease- and isoform-specific propagation characteristics. In exemplary embodiments, tauopathy-derived biological material from distinct diseases converts different Tau isoforms into aggregated Tau assemblies in a seed-dependent manner. FIG. 24A shows experimental results demonstrating that homogenates from Pick's disease (PiD) and progressive supranuclear palsy (PSP) convert 3R and 4R Tau monomers into aggregates, respectively. In embodiments, PiD fibrils and PSP fibrils (Generation 1) amplified using recombinant 3R Tau monomers and recombinant 4R Tau monomers, respectively, under RT-QuIC conditions (e.g., as illustrated in FIG. 29A and FIG. 29C) are sedimented to separate pellet (P) and supernatant (S) fractions and analyzed by SDS-PAGE and Coomassie staining. In embodiments, representative gels are shown for reactions seeded with PiD1-PiD3 homogenates (panel (a)) and for reactions seeded with PSP1-PSP3 homogenates (panel (b)). In embodiments, M denotes a protein marker. In embodiments, 3R Tau migrates at an apparent molecular weight of approximately 52 kDa, and 4R Tau migrates at an apparent molecular weight of approximately 64 kDa. In embodiments, band intensities are quantified to determine the distribution of Tau between pellet and supernatant fractions (e.g., as illustrated in FIG. 29B and FIG. 29D).

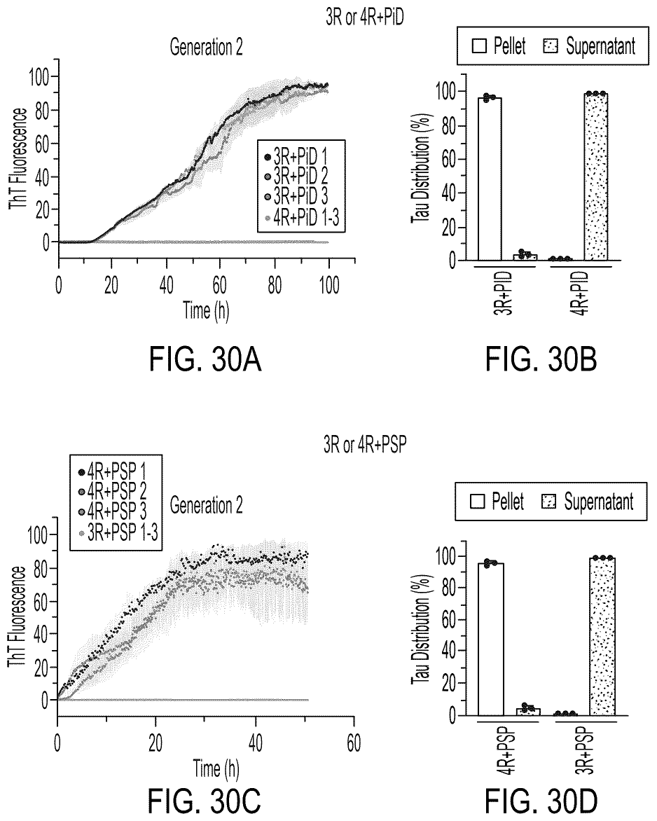

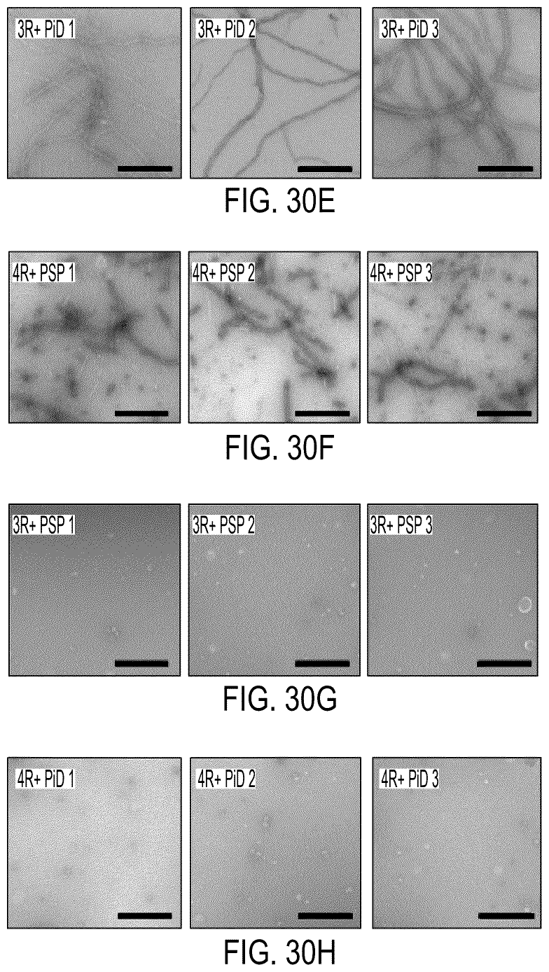

In exemplary embodiments, Tau fibrils derived from distinct tauopathies can be serially amplified while retaining characteristic isoform-specific propagation and cross-seeding properties. FIGS. 24B-1-24B-2 show experimental results demonstrating that Generation 1 Tau fibrils from PiD and PSP can be serially amplified while retaining their characteristic cross-seeding properties. In embodiments, PiD1-PiD3 and PSP1-PSP3 Tau fibrils are serially amplified using recombinant 3R Tau monomers and recombinant 4R Tau monomers, respectively, under RT-QuIC conditions (e.g., as illustrated in FIG. 30A and FIG. 30C). Reaction products are sedimented to separate pellet (P) and supernatant (S) fractions and analyzed by SDS-PAGE and Coomassie staining. In embodiments, representative gels are shown for reactions in which 3R Tau monomers are seeded with PiD fibrils (panel (a)) and in which 4R Tau monomers are cross-seeded with PiD fibrils (panel (b)). In embodiments, representative gels are shown for reactions in which 4R Tau monomers are seeded with PSP fibrils (panel (c)) and in which 3R Tau monomers are cross-seeded with PSP fibrils (panel (d)). In embodiments, M denotes a protein marker, 3R Tau migrates at an apparent molecular weight of approximately 52 kDa, and 4R Tau migrates at an apparent molecular weight of approximately 64 kDa. In embodiments, band intensities are quantified to determine distribution of Tau between pellet and supernatant fractions (e.g., as illustrated in FIG. 30B and FIG. 30D). These illustrative results support various embodiments in which tauopathy-derived Tau assemblies exhibit isoform-selective propagation and seeding barriers that are preserved through serial amplification.

In exemplary embodiments, Tau fibril seeds generated from different tauopathy-derived amplification reactions exhibit reproducible size characteristics suitable for serial propagation. FIGS. 24C-1-24C-2 show experimental results demonstrating that Generation 1 seeds from PiD and PSP have similar lengths. In various embodiments, PiD1-PiD3 and PSP1-PSP3 Tau fibrils generated by amplification (Generation 1) from brain homogenates mixed with recombinant 3R Tau monomers and recombinant 4R Tau monomers, respectively (e.g., as described with reference to FIGS. 29E-29F), are processed to generate seed material by tip sonication for approximately 30 seconds on ice and then imaged by negative-stain transmission electron microscopy (TEM). In embodiments, representative TEM images are shown for seeds generated from 3R Tau+PiD1-PiD3 (panel (a)) with corresponding length quantification (panel (b)), and representative TEM images are shown for seeds generated from 4R Tau+PSP1-PSP3 (panel (c)) with corresponding length quantification (panel (d)). In embodiments, scale bars are 100 nm.

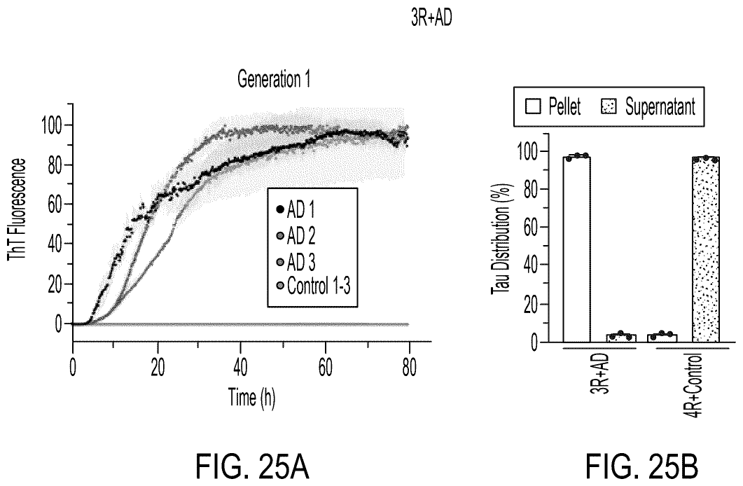

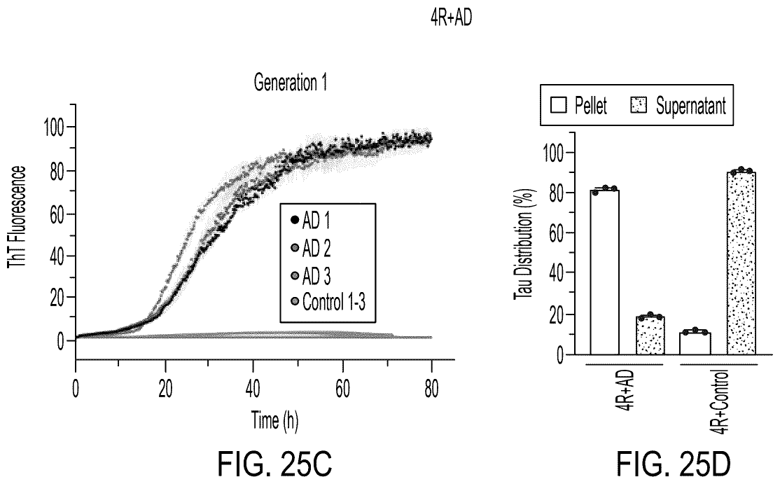

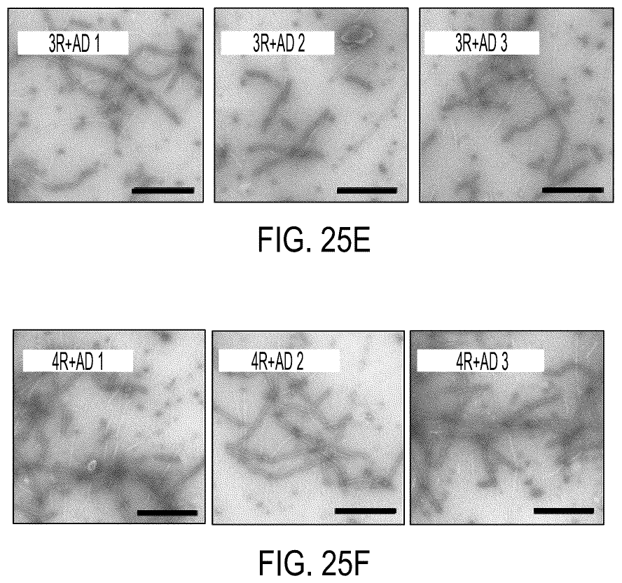

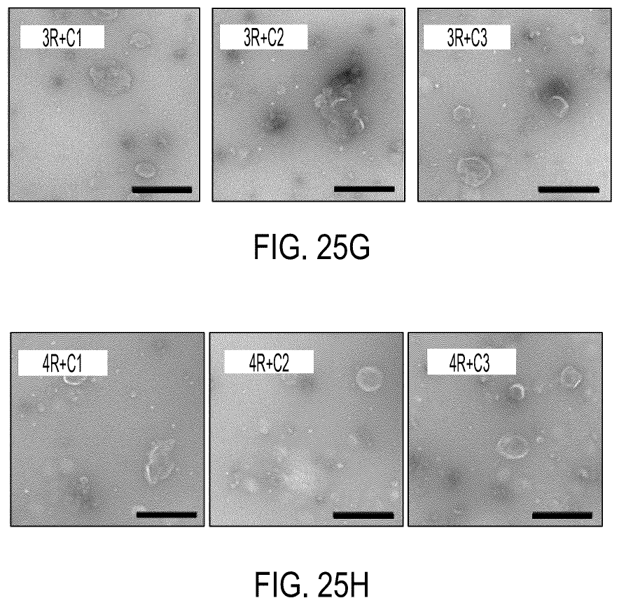

Some additional embodiments and further experimental characterization are shown in FIGS. 25A-30H. FIGS. 25A-25H show experimental results demonstrating that Alzheimer's disease (AD) brain homogenates convert full-length 3R and 4R Tau monomers into aggregated Tau assemblies in the absence of added co-factors, in accordance with various embodiments disclosed herein. In embodiments, tissue extracts from frontal cortex of three separate AD subjects (AD1-AD3) and three non-demented controls (C1-C3) are mixed with recombinant full-length Tau monomers at a concentration of about 10 μM in a low-salt phosphate buffer (e.g., 10 mM sodium phosphate, pH 7.4) under reducing conditions (e.g., in the presence of excess reducing agent such as about 10 mM dithiothreitol). In some embodiments, these reaction conditions are selected to promote a Tau monomer conformation permissive for seed-dependent elongation and to enable co-factor-free propagation. In embodiments, reactions are incubated at about 37° C. using a Real-Time Quaking-Induced Conversion (RT-QuIC) protocol involving intermittent shaking and quiescent incubation to promote fibril fracture and growth, and aggregation is monitored by Thioflavin T (ThT) fluorescence, which increases upon binding β-sheet-rich fibrils.

In various embodiments, FIGS. 25A-25B show that recombinant full-length 3R Tau (e.g., 0N3R) aggregates in the presence of AD homogenates (AD1-AD3) but does not aggregate in the presence of control homogenates (C1-C3), indicating seed-dependent conversion. In some embodiments, the resulting aggregates are referred to as Generation 1 fibrils. In embodiments, completed reactions are sedimented by ultracentrifugation to separate pellet (P) and supernatant (S) fractions, and the fractions are analyzed by SDS-PAGE and Coomassie staining to assess monomer-to-aggregate conversion. In some embodiments, densitometric quantification indicates that in AD-templated reactions more than about 90% of 3R Tau is distributed in the pellet fraction, whereas in control-templated reactions the Tau protein remains predominantly soluble (e.g., about 95%) in the supernatant fraction. In embodiments, the SDS-PAGE gels underlying the sedimentation analyses are shown in FIGS. 18A-18D, for example.

In some embodiments, FIGS. 25C-25D show that recombinant full-length 4R Tau (e.g., 2N4R) similarly aggregates in the presence of AD homogenates (AD1-AD3) but remains largely soluble in the presence of control homogenates (C1-C3), thereby demonstrating that AD-derived seeds can recruit both 3R and 4R Tau isoforms, consistent with mixed-isoform tauopathies. In some embodiments, densitometric quantification indicates that about 80% of 4R Tau is distributed in the pellet fraction in AD-templated reactions, while about 90% remains soluble in the supernatant in control-templated reactions. In embodiments, each biological replicate (AD1-AD3 and C1-C3) is repeated in technical triplicate (n=3), and error bars represent means standard deviation.

In embodiments, FIGS. 25E-25H provide negative-stain transmission electron microscopy (TEM) images of reaction end products following completion of the seeding reactions. In embodiments, fibrils are observed in reactions seeded with AD extracts for both 3R Tau (FIG. 25E) and 4R Tau (FIG. 25F), whereas reactions seeded with control extracts do not show fibrils for 3R Tau (FIG. 25G) or 4R Tau (FIG. 25H), thereby confirming that the aggregates are fibrillar and that spontaneous aggregation is excluded under the disclosed conditions. Scale bars are about 500 nm. Collectively, FIGS. 25A-25H support embodiments in which pathological Tau seeds in AD biological material efficiently template co-factor-free amplification of both 3R and 4R Tau monomeric substrates under defined low-salt and reducing conditions.

FIGS. 26A-1-26D-5 show experimental results demonstrating that added co-factors are not required to serially amplify AD-derived Tau fibrils across multiple amplification generations, in accordance with various embodiments disclosed herein. In embodiments, recombinant Tau monomers (e.g., about 10 μM) are mixed with sonicated AD-derived fibrils (Generation 1) in a low-salt phosphate buffer (e.g., 10 mM sodium phosphate, pH 7.4) at a seed-to-monomer ratio of about 1:100 (i.e., about 1% seed) and incubated at about 37° C. using the RT-QuIC protocol, resulting in formation of Generation 2 fibrils. In embodiments, the amplification steps are repeated to produce additional fibril generations (e.g., Generations 3-6), where for each successive generation about 1% of the end product from the immediately preceding generation is used as seed for the next reaction, thereby resulting in a serial amplification process that successively dilutes the original brain material (e.g., 100-fold dilution per generation).

In some embodiments, the serial amplification design results in progressive dilution of non-Tau components from the original brain homogenate (e.g., starting from a total protein concentration of about 30 μg/mL in Generation 1), such that by Generation 6 the original brain-derived material is diluted to an overall dilution on the order of about 1×10\hat{ }-10 relative to the recombinant Tau substrate. In embodiments, this demonstrates that at advanced generations the concentration of any brain-derived co-factors is negligible, yet amplification continues, thereby supporting that Tau assemblies themselves are sufficient to drive propagation under the disclosed conditions.

In embodiments, FIGS. 26A-1-26A-5 and 26B-1-2-26B-5 show ThT kinetics and sedimentation analyses for serial amplification reactions using 3R Tau monomers. In some embodiments, ThT fluorescence traces demonstrate seeded growth across multiple generations, and sedimentation-densitometric analyses show efficient conversion of soluble 3R Tau monomers to aggregated states, with about 90% of Tau protein distributed in pellet fractions. In embodiments, the SDS-PAGE gels underlying the sedimentation analyses for seeded reactions with 3R Tau monomers are shown in FIG. 19A-1-19A-2, for example.

In embodiments, FIGS. 26C-1-26C-5 and 27D-1-27D-5 show ThT kinetics and sedimentation analyses for serial amplification reactions using 4R Tau monomers. In some embodiments, ThT fluorescence traces demonstrate seeded growth across multiple generations, and sedimentation-densitometric analyses show efficient conversion of soluble 4R Tau monomers to aggregated states in the absence of added co-factors. In embodiments, the SDS-PAGE gels underlying the sedimentation analyses for seeded reactions with 4R Tau monomers are shown in FIGS. 19B-1-19B-2, for example. In embodiments, AD1-AD3 reflect reactions with seeds from the immediately preceding fibril generation, and the seeds used for serial amplification descend from aggregates present in the original AD brain homogenates. In embodiments, each biological replicate is repeated in technical triplicate (n=3), and error bars represent means±standard deviation.

In some embodiments, prior to initiating serial amplification reactions, fibrils from Generation 1 are sonicated to generate seed material, and negative-stain TEM inspection confirms short fibrillar seeds with an average length in the range of about 63 to about 68 nm (e.g., as shown in FIGS. 23A-23D). In some embodiments, TEM analyses of the last amplified generation (e.g., Generation 6) confirm that Tau aggregates maintain fibrillar morphology following serial propagation (e.g., as shown in FIG. 19C). Collectively, FIGS. 26A-1-26D-5 support embodiments in which co-factor-free serial amplification can be repeated over multiple generations to generate large quantities of Tau assemblies while progressively diluting non-Tau components from the initial biological source.

FIG. 27 shows experimental results demonstrating that cross-seeding abilities of AD-derived Tau fibrils are preserved after serial amplification, in accordance with various embodiments disclosed herein. In embodiments, recombinant Tau monomers (e.g., about 10 M) are mixed with Generation 6 seeds composed of the other Tau isoform at a seed-to-monomer ratio of about 1:100 and incubated at about 37° C. using RT-QuIC conditions. In embodiments, 4R Tau monomers are incubated with 3R Tau seeds and, conversely, 3R Tau monomers are incubated with 4R Tau seeds, thereby evaluating whether serial amplification affects hallmark AD cross-seeding behavior.

In embodiments, FIG. 27 (top row) shows ThT kinetics and sedimentation analyses of 4R Tau monomers grown onto 3R Tau seeds. In embodiments, FIG. 27 (bottom row) shows ThT kinetics and sedimentation analyses of 3R Tau monomers grown onto 4R Tau seeds. In some embodiments, ThT fluorescence traces demonstrate robust seeded growth in both cross-seeding directions, and sedimentation analyses demonstrate recruitment of Tau monomers into aggregated pellet fractions, indicating preservation of cross-seeding capability. In embodiments, SDS-PAGE gels underlying the sedimentation analyses are shown in FIGS. 20A-20B, for example. In embodiments, each biological replicate is repeated in technical triplicate (n=3), and error bars represent means±standard deviation. In some embodiments, differences in kinetics between cross-seeding directions may be observed, while overall recruitment and conversion remain robust. Collectively, FIG. 27 supports embodiments in which serially amplified AD-derived Tau assemblies retain disease-relevant propagation characteristics, including cross-seeding between 3R and 4R Tau isoforms.

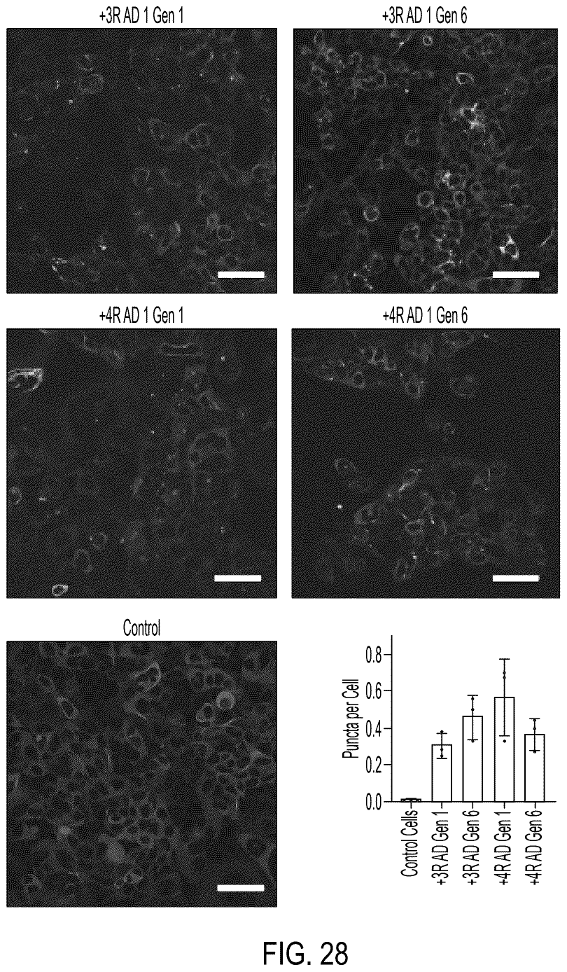

FIG. 28 shows experimental results demonstrating that Tau fibrils amplified from AD brain homogenates induce intracellular Tau aggregation, in accordance with various embodiments disclosed herein. In embodiments, monoclonal HEK293 cells expressing htau40P301S (a 2N4R Tau variant) tagged with enhanced yellow fluorescent protein (EYFP) at the C-terminus are transfected with Tau seeds and incubated for about 24 hours at about 37° C. In embodiments, Tau seeds derived from serial amplification reactions (e.g., sonicated reaction end products) are introduced into cells to evaluate seeding propensity and functional activity of the amplified Tau assemblies.

In embodiments, FIG. 28 (top row and middle row) shows representative fluorescence microscopy images of cells transfected with AD1 seeds amplified using 3R Tau monomers (FIG. 28 (top row)) or 4R Tau monomers (FIG. 28 (middle row)), comparing Generation 1 seeds (left panels) versus Generation 6 seeds (right panels). In embodiments, cells transfected in the absence of seeds serve as controls (FIG. 28 (Control)). In some embodiments, externally introduced Tau seeds, regardless of isoform composition or amplification generation, induce formation of intracellular puncta indicative of seeded Tau aggregation, whereas buffer controls do not. Scale bars are about 40 m.

In embodiments, FIG. 28 (bar graph) provides quantification of puncta per cell for each biological replicate (AD1-AD3), with error bars representing means±standard deviation. In some embodiments, puncta counts are similar across seed types and generations, indicating that serial amplification does not eliminate functional seeding competence.

TABLE 1

| Seeds | Origin | Number of Cells | Number of Puncta |
|---|---|---|---|
| 3R Tau Generation 1 | AD 1 | 922 | 262 |
| | AD 2 | 944 | 359 |
| | AD 3 | 1184 | 300 |
| 4R Tau Generation 1 | AD 1 | 551 | 181 |
| | AD 2 | 855 | 580 |
| | AD 3 | 1058 | 744 |
| 3R Tau Generation 6 | AD 1 | 1151 | 373 |
| | AD 2 | 1540 | 862 |
| | AD 3 | 1292 | 647 |
| 4R Tau Generation 6 | AD 1 | 841 | 369 |
| | AD 2 | 802 | 218 |
| | AD 3 | 573 | 228 |
| Buffer Control 1 | | 690 | 7 |
| Buffer Control 2 | | 573 | 8 |
| Buffer Control 3 | | 747 | 6 |

In some embodiments, Tau assemblies produced by serial amplification are evaluated in a cell-based seeding assay. In an exemplary implementation, amplified Tau fibril seeds are introduced into HEK293 cells expressing a Tau reporter construct, and seeded aggregation is assessed by puncta formation. Quantification may include counting a number of cells analyzed and a number of intracellular puncta observed, and calculating puncta-per-cell values. Table 1 summarizes exemplary cell and puncta counts for replicate experiments corresponding to FIG. 28 (top row, middle row, and Control) and FIG. 21A-21D, and the summarized counts were used to generate puncta-per-cell values shown in FIG. 28 (bar graph).

FIGS. 29A-29F shows experimental results demonstrating that co-factors are not required to amplify Tau filaments from Pick's disease (PiD) and progressive supranuclear palsy (PSP) brain homogenates, in accordance with various embodiments disclosed herein. In embodiments, recombinant Tau monomers (e.g., about 10 μM) are mixed with PiD brain homogenates (PiD1-PiD3) or PSP brain homogenates (PSP1-PSP3) (e.g., about 30 g/mL total brain protein) in low-salt phosphate buffer (e.g., 10 mM sodium phosphate, pH 7.4) and incubated at about 37° C. using the RT-QuIC protocol, resulting in formation of Generation 1 fibrils.

In embodiments, FIGS. 29A-29B show ThT kinetics and sedimentation analyses of PiD-seeded reactions with 3R Tau monomers, demonstrating time-dependent ThT fluorescence increases and distribution of the majority of Tau protein into pellet fractions, consistent with fibril formation. In embodiments, FIGS. 29C-29D show ThT kinetics and sedimentation analyses of PSP-seeded reactions with 4R Tau monomers, similarly demonstrating time-dependent ThT fluorescence increases and prominent distribution of Tau protein into pellet fractions. In embodiments, SDS-PAGE gels underlying the sedimentation analyses are shown in FIG. 24A, for example. In embodiments, each biological replicate is repeated in technical triplicate (n=3), and error bars represent means±standard deviation.

In embodiments, FIGS. 29E-29F provide representative negative-stain TEM images confirming that the aggregates formed are filamentous rather than amorphous. In embodiments, long filaments are observed for PiD-seeded reactions with 3R Tau monomers (FIG. 29E) and PSP-seeded reactions with 4R Tau monomers (FIG. 29F). Scale bars are about 500 nm. Collectively, FIGS. 29A-29F support embodiments in which tauopathy-specific seeds derived from PiD and PSP biological materials can template co-factor-free amplification of full-length Tau monomers in an isoform-consistent manner.

FIGS. 30A-30H shows experimental results demonstrating that Generation 1 Tau fibrils from PiD and PSP can be serially amplified while retaining their characteristic isoform-selective propagation and cross-seeding barrier properties, in accordance with various embodiments disclosed herein. In embodiments, recombinant Tau monomers (e.g., about 10 μM) are mixed with sonicated PiD- or PSP-derived fibrils (Generation 1) in low-salt phosphate buffer (e.g., 10 mM sodium phosphate, pH 7.4) at a seed-to-monomer ratio of about 1:100 and incubated at about 37° C. using RT-QuIC conditions. In embodiments, for homotypic seeding reactions, this results in formation of Generation 2 fibrils, while for heterotypic reactions a robust seeding barrier is observed.

In embodiments, FIGS. 30A-30B show ThT kinetics and sedimentation analyses for PiD-seeded reactions, demonstrating that PiD-derived seeds support recruitment and conversion of 3R Tau monomers, while 4R Tau monomers do not convert in heterotypic reactions. In embodiments, FIGS. 30C-30D show ThT kinetics and sedimentation analyses for PSP-seeded reactions, demonstrating that PSP-derived seeds support recruitment and conversion of 4R Tau monomers, while 3R Tau monomers do not convert in heterotypic reactions. In embodiments, SDS-PAGE gels underlying the sedimentation analyses for Generation 1 seeded reactions are shown in FIG. 24B-1-24B-2, for example. In embodiments, each biological replicate is repeated in technical triplicate (n=3), and error bars represent means±standard deviation.

In embodiments, FIGS. 30E-30H provide representative negative-stain TEM images of reaction end products confirming fibril formation in homotypic reactions and absence of fibrils in heterotypic reactions. In embodiments, fibrils are observed for 3R Tau+PiD1-PiD3 (FIG. 30E) and 4R Tau+PSP1-PSP3 (FIG. 30F). In embodiments, heterotypic reactions produce no fibrils, including 4R Tau+PiD (FIG. 30G) and 3R Tau+PSP1-PSP3 (FIG. 30H). Scale bars are about 500 nm. In embodiments, PiD1-PiD3 and PSP1-PSP3 reflect reactions with seeds from Generation 1 fibrils, and these seeds descend from aggregates in the original brain homogenates. Collectively, FIGS. 30A-30H support embodiments in which tauopathy-derived seeds preserve isoform-specific recruitment and seeding barrier behavior through amplification, consistent with the isoform-specific composition of Tau fibrils in PiD and PSP.

TABLE 2

| Subject | Age | Gender | Postmortem Interval (h) |
|---|---|---|---|
| Control 1 | 61 | M | 16 |
| Control 2 | 59 | M | 16.3 |
| Control 3 | 56 | F | 17.3 |
| AD 1 | 79 | M | 18 |
| AD 2 | 84 | M | 11 |
| AD 3 | 80 | M | 9.8 |
| PiD 1 | 73 | M | 12.4 |
| PiD 2 | 72 | M | 4.5 |
| PiD 3 | 76 | F | 3.3 |
| PSP 1 | 71 | F | 5.3 |
| PSP 2 | 66 | M | 8.5 |
| PSP 3 | 71 | M | 7 |

Table 2 provides exemplary demographic and postmortem metadata for tauopathy and control brain tissue cases used in certain experimental embodiments described herein (e.g., the embodiments corresponding to FIGS. 25A-30H). Use of multiple independent cases and inclusion of control tissue sources support that the observed co-factor-free serial amplification is seed-dependent and reproducible across distinct biological samples.

The examples disclosed herein are provided to illustrate certain embodiments of the present disclosure and are not intended to limit the scope of the disclosure. For example, unless otherwise indicated, reagents, instruments, and conditions described below are exemplary and may be substituted with equivalent materials and equipment. In some embodiments, the steps described herein may be modified, reordered, scaled, automated, or combined.

In some embodiments, recombinant Tau monomeric substrates are produced from DNA constructs encoding human Tau isoforms. By way of example, gene-optimized DNA inserts encoding human wildtype 0N3R Tau and 2N4R Tau may be synthesized and cloned into expression vectors. In certain embodiments, inserts are cloned into pET-based expression vectors using restriction sites such as NcoI and XhoI. In some embodiments, one or more stop codons are included to eliminate affinity tags.

In some embodiments, recombinant Tau proteins are expressed in bacterial host cells and purified prior to use in amplification reactions.

By way of example, BL21 (DE3) E. coli cells may be transformed with expression vectors encoding 0N3R Tau (3R Tau) or 2N4R Tau (4R Tau), plated on antibiotic-containing agar, and expanded in liquid culture. In certain embodiments, cultures are grown at approximately 37° C. with shaking until reaching an optical density (OD600) of approximately 0.7-1.0, followed by induction of protein expression with an inducer such as isopropyl β-D-1-thiogalactopyranoside (IPTG) (e.g., about 0.5 mM). In some embodiments, cells are harvested after an additional incubation period (e.g., about 1-6 hours).

In some embodiments, harvested cells are resuspended in a lysis buffer including one or more chelating agents and reducing agents (e.g., EDTA and β-mercaptoethanol), salt (e.g., NaCl), and a buffering agent (e.g., Pipes at pH about 6-7). In certain embodiments, lysates are subjected to heating (e.g., about 70-90° C.) and mechanical disruption (e.g., sonication). Soluble Tau may be separated from insoluble material by centrifugation.

In some embodiments, Tau is precipitated (e.g., with ammonium sulfate), collected, and resolubilized in a reducing buffer (e.g., DTT-containing buffer). In certain embodiments, Tau is purified using ion exchange chromatography (e.g., Mono S) and/or size exclusion chromatography (e.g., Superdex 200). Fractions may be evaluated by SDS-PAGE with Coomassie staining and pooled based on Tau content and purity.

In some embodiments, purified Tau is transferred into dialysis tubing and dialyzed against a phosphate buffer (e.g., sodium phosphate, pH about 7-8) optionally including a reducing agent. In some embodiments, purified Tau is aliquoted, flash-frozen, and stored at low temperature (e.g., −80° C.) until use.

In some embodiments, Tau seed material is derived from biological samples obtained from subjects affected by tauopathies, as well as control samples. By way of example, postmortem brain tissue may be obtained from cases including Alzheimer's disease (AD), Pick's disease (PiD), progressive supranuclear palsy (PSP), and non-demented controls. In some embodiments, multiple biological replicates per condition are used.

In some embodiments, neuropathological evaluation is performed according to established staging paradigms. In some embodiments, demographic variables (e.g., age, sex, postmortem interval) do not materially affect amplification outcomes.

In some embodiments, frozen human brain tissue is processed to obtain homogenates containing Tau seed material.

By way of example, tissue may be combined at approximately a 1:10 (w/v) ratio with a homogenization buffer including a buffering agent (e.g., HEPES at pH about 7-8), chelator (e.g., EDTA), salt (e.g., NaCl), detergent (e.g., Triton X-100), and one or more protease inhibitors. In some embodiments, homogenization is performed on ice using a tissue grinder, followed by sonication (e.g., bath sonication), centrifugation (e.g., about 1,000-10,000 g), and collection of supernatants.

In some embodiments, total protein concentration is determined (e.g., by BCA assay), and homogenates are normalized to a selected concentration (e.g., about 1-10 mg/mL, such as about 3 mg/mL), aliquoted, flash-frozen, and stored at low temperature until use.

In some embodiments, Tau assemblies are amplified by contacting recombinant monomeric Tau with seed-containing biological material under co-factor-free conditions.

By way of example, recombinant 3R Tau monomers or 4R Tau monomers may be combined with brain homogenates from AD, PiD, PSP, or control cases. In certain embodiments, homogenates are diluted (e.g., about 1:10 to about 1:1,000) to a final total protein concentration (e.g., about 30 μg/mL). In some embodiments, monomeric Tau is included at a concentration of about 0.1 μM to about 1 mM, such as about 1 μM to about 100 M, for example about 10 μM.

In some embodiments, reactions are performed in a low ionic strength buffer, such as a phosphate buffer (e.g., about 10 mM sodium phosphate at pH about 7.4). In some embodiments, reactions are performed under reducing conditions, such as in the presence of a reducing agent (e.g., DTT, such as about 10 mM). In some embodiments, Thioflavin T (ThT) is included as a reporter dye (e.g., about 0.1-50 M, such as about 5 μM). In some embodiments, total reaction volume is about 10 μL to about 10 mL, such as about 400 μL.

In some embodiments, the reaction mixture is subjected to intermittent agitation and incubation in a microplate format. By way of example, the mixture may be dispensed into multiwell plates (e.g., 96-well plates), sealed, and monitored in a plate reader. In certain embodiments, the plate reader is configured to perform intermittent shaking (e.g., shaking for about 0.1-10 minutes every about 1-60 minutes, such as shaking for about 1 minute every 10 minutes) at a selected speed (e.g., about 100-1,000 rpm, such as about 400 rpm). In some embodiments, fluorescence is monitored over time using excitation/emission wavelengths suitable for ThT (e.g., excitation ~440 nm; emission ~480 nm).

In some embodiments, reactions seeded with AD homogenates convert both 3R and 4R Tau monomers into fibrillar Tau assemblies. In some embodiments, reactions seeded with PiD homogenates preferentially convert 3R Tau monomers. In some embodiments, reactions seeded with PSP homogenates preferentially convert 4R Tau monomers. In some embodiments, control homogenates do not seed conversion under the selected conditions, thereby indicating seed-dependent amplification.

In some embodiments, one or more control experiments are performed to evaluate reaction dependencies.

In some embodiments, increasing ionic strength inhibits amplification. By way of example, NaCl may be added to the reaction buffer (e.g., about 150 mM NaCl), and ThT fluorescence and/or sedimentation analysis may show reduced or absent seeded amplification relative to low-salt conditions.

In some embodiments, oxidation of Tau monomers inhibits recruitment onto pathological seeds. In some embodiments, oxidized 3R Tau dimers or compact oxidized 4R Tau monomers are used in amplification reactions without reducing agent, resulting in reduced or absent amplification.

In some embodiments, Tau monomers incubated without seed material do not substantially aggregate under the amplification conditions, supporting that amplification is seed-dependent.

In some embodiments, Tau assemblies produced in Generation 1 are processed to generate seed material for subsequent amplification cycles.

In some examples, Generation 1 reaction products may be recovered, and technical replicates may be pooled. In some embodiments, samples are diluted into a buffer (e.g., phosphate buffer) and subjected to mechanical disruption (e.g., tip sonication) to fragment assemblies into seed-competent fibril fragments. In some embodiments, sonication is performed for about 1-120 seconds (e.g., about 30 seconds) on ice using a probe sonicator.

In some embodiments, fragmented seeds are diluted into fresh reaction buffer containing additional recombinant Tau monomers. In certain embodiments, seeds are introduced at a seed-to-monomer ratio of about 0.001% to about 20%, such as about 1% (e.g., 1:100). The amplification assay is repeated using conditions similar to those used in Generation 1, resulting in Generation 2 assemblies.

In some embodiments, this process is repeated iteratively to generate Generation 3, Generation 4, Generation 5, Generation 6, and additional generations. In certain embodiments, each amplification generation results in an approximately 100-fold dilution of the original biological homogenate-derived material. In some embodiments, serial amplification yields Tau assemblies that remain propagative across multiple generations without addition of negatively charged co-factors.

In some embodiments, cross-seeding barriers are evaluated by using monomeric Tau isoforms different from the isoforms present in the seeds.

By way of example, 4R Tau monomers may be incubated with 3R Tau fibril seeds and/or 3R Tau monomers may be incubated with 4R Tau fibril seeds. In some embodiments, AD-derived assemblies exhibit cross-seeding, including recruitment of 3R Tau onto 4R Tau assemblies and recruitment of 4R Tau onto 3R Tau assemblies. In some embodiments, PiD-derived assemblies preferentially recruit 3R Tau and show a barrier to 4R Tau recruitment, and PSP-derived assemblies preferentially recruit 4R Tau and show a barrier to 3R Tau recruitment.

In some embodiments, conversion of Tau monomers into aggregated assemblies is quantified by sedimentation.

By way of example, after an amplification round, samples may be centrifuged (e.g., ultracentrifugation at about 100,000 g to about 500,000 g, such as about 400,000 g) for a selected duration (e.g., about 5-120 minutes, such as about 30 minutes) at low temperature (e.g., about 4° C.) to separate pellet and supernatant fractions. Fractions may be analyzed by SDS-PAGE and Coomassie staining.

In some embodiments, band intensities are quantified (e.g., using image analysis software) to determine the fraction of Tau in pellet versus supernatant, thereby providing a quantitative measure of conversion.

In some embodiments, Tau monomers are oxidized to generate disulfide-linked species.

By way of example, Tau may be incubated with an oxidizing agent (e.g., hydrogen peroxide, such as about 1 mM) for a selected period (e.g., about 1-48 hours, such as about 24 hours) at ambient temperature (e.g., about 22° C.) in a buffer (e.g., HEPES buffer at pH about 8.2) containing salt (e.g., about 100 mM NaCl). In some embodiments, oxidation yields 3R Tau dimers via intermolecular disulfide bonds and/or compact 4R Tau monomers via intramolecular disulfide bonds.

In some embodiments, oxidized species are purified (e.g., by size exclusion chromatography) and dialyzed into a buffer compatible with amplification reactions. In some embodiments, oxidation state is confirmed by electrophoretic analysis, including non-reducing SDS-PAGE and/or native gel electrophoresis.

In some embodiments, amplified Tau assemblies are evaluated by electron microscopy.

By way of example, reaction mixtures may be diluted to a selected concentration (e.g., about 0.1-20 μM, such as about 5 μM) and applied to grids for negative stain TEM imaging. In some embodiments, TEM confirms fibrillar morphology of assemblies produced by seed-dependent amplification and/or serial amplification.

While the disclosure above discusses details of exemplary embodiments of a multistep Tau amplification system and associated processes, additional modifications may be contemplated and are considered to be a part of the present disclosure. For example, one or more of the components of multistep Tau amplification system 1700 of FIG. 17 may be automated using, for example, a robot system such that at least a portion of multistep Tau amplification system 1700 may be fully automated. Alternative methods of providing the quaking and/or fibril breakage may be used in place of those discussed above.

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of various embodiments of any apparatus embodiment, a method or process embodiment, or even merely a variation of any element of these. Particularly, it should be understood that the words for each element may be expressed by equivalent apparatus terms or method terms, even if only the function or result is the same. Such equivalent, broader, or even more generic terms should be considered to be encompassed in the description of each element or action. Such terms can be substituted where desired to make explicit the implicitly broad coverage to which this disclosure is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “protrusion” should be understood to encompass disclosure of the act of “protruding,” whether explicitly discussed or not, and, conversely, were there only disclosure of the act of “protruding,” such a disclosure should be understood to encompass disclosure of a “protrusion.” Such changes and alternative terms are to be understood to be explicitly included in the description.

1. A method for serial amplification of Tau filaments, comprising:
(a) contacting monomeric Tau protein with Tau seed material under conditions permissive for seed-dependent fibril elongation to produce amplified Tau filaments;
(b) processing at least a portion of the amplified Tau filaments to generate Tau seed material for further amplification; and
(c) subjecting the generated Tau seed material to one or more additional amplification steps by repeating steps (a) and (b), thereby producing serially amplified Tau filaments,
wherein the monomeric Tau comprises one or more Tau isoforms, variants, fragments, or modified forms, comprising 3R Tau, 4R Tau, 0N Tau, 1N Tau, or 2N Tau isoforms, truncated Tau fragments, recombinant Tau constructs, post-translationally modified Tau, disease-associated Tau variants, or any combinations thereof.

2. The method of claim 1, wherein the Tau fibril seed material is derived from a Tauopathy.

3. The method of claim 1, wherein the monomeric Tau comprises 3R Tau isoforms, 4R Tau isoforms, or combinations thereof.

4. The method of claim 1, wherein the serial amplification comprises between 2 and 50 amplification cycles, each cycle diluting the Tau seed material such that the serially amplified Tau filaments are substantially free of non-Tau components originating from a biological source of the Tau fibril seed material.

5. The method of claim 1, wherein amplification is performed under shaking, agitation, quiescent incubation, sonication, mechanical cycling, temperature cycling, or RT-QuIC-like conditions in the absence of added negatively charged co-factors that promote Tau aggregation.

6. The method of claim 1, wherein the serially amplified Tau filaments preserve at least one structural or functional feature of the Tau fibril seed material.

7. The method of claim 1, wherein the serially amplified Tau filaments exhibit structural divergence relative to the Tau fibril seed material.

8. The method of claim 1, further comprising characterizing the amplified Tau filaments using one or more techniques selected from fluorescence kinetics, biochemical sedimentation, protease digestion profiling, mass spectrometry, electron microscopy, atomic force microscopy, or cell-based seeding assays wherein the characterization demonstrates substantial conversion of Tau monomers into filamentous assemblies.

9. The method of claim 1, wherein amplification is conducted in a microplate, tube, microfluidic device, continuous-flow system, or automated amplification platform.

10. The method of claim 1, wherein the monomeric Tau is phosphorylated, acetylated, truncated, recombinant, or chemically modified.

11. A method for identifying a compound that modulates Tau assembly propagation, comprising:
(a) contacting monomeric Tau protein with Tau seed material under conditions permissive for seed-dependent Tau assembly growth to produce amplified Tau assemblies, wherein the monomeric Tau comprises one or more Tau isoforms, variants, fragments, or modified forms;
(b) processing at least a portion of the amplified Tau assemblies to generate Tau seed material for further amplification;
(c) repeating steps (a) and (b) one or more times to produce serially amplified Tau assemblies;
(d) performing the serial amplification of steps (a)-(c) in the presence of a test compound;
(e) performing the serial amplification of steps (a)-(c) in the absence of the test compound as a control; and
(f) comparing one or more propagation characteristics of the Tau assemblies produced in steps (d) and (e),
wherein the test compound is identified as modulating Tau assembly propagation based on a difference in at least one propagation characteristic selected from amplification kinetics, assembly morphology, seeding activity, biochemical properties, or cellular propagation behavior.

12. The method of claim 11, wherein the test compound is selected from small molecules, peptides, antibodies, nucleic acids, metabolites, or post-translational modification inhibitors.

13. The method of claim 11, wherein modulation of Tau assembly propagation is quantified by changes in lag time, elongation rate, fibril mass, protease resistance, or fibril ultrastructure.

14. The method of claim 11, wherein the method is conducted in a high-throughput or automated multiwell screening format.

15. The method of claim 11, wherein the test compound modulates Tau assembly propagation by interacting with Tau monomers, Tau assemblies, or Tau assembly ends.

16. A method for serial amplification of Tau assemblies, comprising:
(a) contacting monomeric Tau protein with Tau seed material obtained from a biological sample under conditions permissive for seed-dependent Tau assembly growth to produce amplified Tau assemblies;
(b) processing at least a portion of the amplified Tau assemblies to generate Tau seed material for further amplification; and
(c) subjecting the generated Tau seed material to one or more additional amplification steps by repeating steps (a) and (b), thereby producing serially amplified Tau assemblies,
wherein the Tau assemblies comprise Tau species capable of templated propagation, and wherein the monomeric Tau comprises one or more Tau isoforms, variants, fragments, or modified forms.

17. The method of claim 16, wherein the Tau seed material is obtained by homogenization and centrifugation of the biological sample.

18. The method of claim 16, wherein the biological sample comprises cerebrospinal fluid, brain homogenate, blood-derived extracellular vesicles, or postmortem tissue.

19. The method of claim 16, wherein the serially amplified Tau assemblies are used to differentiate Tau conformational strains or Tauopathy subtypes.

20. The method of claim 16, wherein the serially amplified Tau assemblies are used for structural, biochemical, or functional analysis.
